(19) United States
(12) Patent Application Publication
(10) Pub. No.: US 2026/0114957 A1
(43) Pub. Date: Apr. 30, 2026

(54) Method, apparatus, and computer-readable recording medium for providing orthodontic status and orthodontic treatment evaluation information based on dental scan data of patient

(71) Applicant: INNODTECH, Inc., Gwangju (KR)

(72) Inventor: Bo Hoon JOO, Seoul (KR)

(21) Appl. No.: 18/294,374

(22) PCT Filed: Aug. 10, 2022
(86) PCT No.: PCT/KR2022/011900

§ 371 Date: Feb. 1, 2024

(30) Foreign Application Priority Data
Aug. 10, 2021 (KR) 10-2021-0105222
Aug. 10, 2021 (KR) 10-2021-0105223

(51) Int. Cl. A61C 7/00; G06T 7/00

(52) U.S. Cl. CPC A61C 7/002; G06T 7/0014; G06T 2207/30036

(57) ABSTRACT

The present invention provides a method for providing an orthodontic status and orthodontic treatment evaluation information based on dental scan data of a patient, implemented in a computing device including one or more processors and one or more memories storing instructions executable by the processors. The method is characterized by comprising: an initial image acquisition step of, when first dental scan data, which is three-dimensional scan data acquired by photographing the patient's head, is received, acquiring a first dental image, which is an image of the patient's teeth arrangement, on the basis of the received first dental scan data; a correction image acquisition step of, when the acquisition of the first dental image is completed, confirming a teeth arrangement status based on the first dental image through a pre-stored algorithm, and acquiring treatment solution information for correcting the teeth arrangement based on the confirmed teeth arrangement state, wherein acquired is a second dental image, which is an image of predicted teeth arrangement upon completion of the correction based on the acquired treatment solution information; an orthodontic appliance design creation step of, when the acquisition of the second dental image is completed, creating a design of a transparent orthodontic appliance for correcting the patient's teeth arrangement into a teeth arrangement corresponding to the second dental image; an intermediate image acquisition step of, in the process of correcting the patient's teeth arrangement as the patient has on the transparent orthodontic appliance based on the created design, when second dental scan data, which is new three-dimensional scan data, is received, acquiring a third dental image, which is an image of the patient's teeth arrangement being corrected, on the basis of the received second dental scan data; and an orthodontic status information provision step of, when the acquisition of the third dental image is completed, acquiring teeth movement vector information of the patient through the first dental image, the second dental image, and the third dental image, and generating orthodontic status information for orthodontic treatment of the patient on the basis of the acquired teeth movement vector information and providing same to a medical personnel account.

### TECHNICAL FIELD

The present invention relates to a method for providing an orthodontic status and orthodontic treatment evaluation information based on tooth part scan data of a patient, and more specifically, to a technology in which an image for patient's teeth arrangement is acquired based on tooth part scan data acquired by capturing a patient's head, a type of patient's malocclusion is confirmed based on the acquired image for patient's teeth arrangement, treatment solution information corresponding to the confirmed type of malocclusion and an image for predicted teeth arrangement upon orthodontic completion are acquired, a design of a transparent orthodontic appliance is generated, an image for arrangement of the patient's teeth that are being corrected is acquired when new tooth part scan data is acquired during orthodontic treatment so as to determine orthodontic treatment status for teeth arrangement, a type of malocclusion for patient's teeth is determined based on the tooth part scan data, an orthodontic target value is acquired based on the treatment solution information for correcting the determined malocclusion, and an orthodontic achievement value for each of the teeth to be corrected is acquired based on second tooth part scan data received later so as to acquire orthodontic treatment evaluation information, which is evaluation information about the orthodontic treatment, based on the orthodontic achievement value and the orthodontic target value.

### BACKGROUND ART

The market size of the world's transparent orthodontic appliances has expanded to a compound annual growth rate (CAGR) of 23.1%, and is expected to reach $6 billion by 2027. As technology advances and the demand for customized transparent orthodontic appliances effective for user teeth arrangement increase, market growth is being accelerated. Accordingly, various technologies are being developed in line with this trend in the dental industry, and representatively, a technology for confirming the progress of patient's teeth arrangement is being developed.

As an example, Korean Unexamined Patent Publication No. 10-2015-0039028 (an orthodontic simulation method and a system for performing the same) discloses a technology of rearranging individual teeth by setting a virtual position of the teeth to be close to adjacent teeth individual teeth are corrected while being identified from a two-dimensional teeth image.

However, the related art described above merely discloses a technology of virtually setting the position of teeth close to adjacent teeth after the orthodontic treatment and rearranging the position of the teeth, and does not disclose a technology of determining which type of malocclusion the patient's teeth arrangement is based on patient's tooth part scan data, and acquiring treatment solution information suitable for the determined malocclusion and an image for predicted teeth arrangement upon orthodontic completion to generate a design of a transparent orthodontic appliance customized to the patient's teeth arrangement, and a technology of acquiring an image for arrangement of the teeth that are being corrected when new tooth part scan data is acquired during a treatment process to determine a current status of orthodontic treatment for the patient's teeth arrangement, and thus, there is a need for a technology capable of solving the above problems.

### DISCLOSURE

### Technical Problem

Accordingly, an object of the present invention is to provide a transparent orthodontic appliance suitable for a state of patient's teeth arrangement by acquiring an image for the patient's teeth arrangement based on tooth part scan data acquired by capturing a patient's head, confirming a type of malocclusion of a patient based on the acquired image for the patient's teeth arrangement, acquiring treatment solution information corresponding to the confirmed type of malocclusion and an image for predicted teeth arrangement upon orthodontic completion, generating a design of the transparent orthodontic appliance, acquiring an image for arrangement of patient's teeth being corrected when new tooth part scan data is acquired during orthodontic treatment, and determining orthodontic treatment status for the teeth arrangement, and is to provide the patient with reliability of the orthodontic treatment of the patient's teeth through orthodontic treatment status information.

### Technical Solution

According to one embodiment of the present invention, there is provided a method for providing an orthodontic status and orthodontic treatment evaluation information based on tooth part scan data of a patient, which is implemented by a computing device including one or more processors and one or more memories storing instructions executable by the processors, which includes: an initial image acquisition step of acquiring a first tooth image, which is an image for patient's teeth arrangement, based on received first tooth part scan data when first tooth part scan data, which is three-dimensional scan data acquired by capturing a patient's head, is received; an orthodontic image acquisition step of, when the acquisition of the first tooth part image is completed, confirming a teeth arrangement state based on the first tooth image through a pre-stored algorithm, acquiring treatment solution information for correcting the teeth arrangement based on the confirmed teeth arrangement state, and acquiring a second tooth image, which is an image for predicted teeth arrangement upon orthodontic completion based on the acquired treatment solution information; an orthodontic appliance design generation step of, when the acquisition of the second tooth image is completed, generating a design of a transparent orthodontic appliance for correcting the patient's teeth arrangement into teeth arrangement corresponding to the second tooth image; an intermediate image acquisition step of acquiring a third tooth image, which is an image for arrangement of patient's teeth being corrected, based on received second tooth part scan data when second tooth part scan data, which is new three-dimensional scan data, is received in a process of correcting the patient's teeth arrangement as the patient wears the transparent orthodontic appliance based on the generated design; and an orthodontic status information provision step of, when the acquisition of the third tooth image is completed, acquiring tooth movement vector information about the patient through the first tooth image, the second tooth image, and the third tooth image to generate orthodontic status information for orthodontic treatment of the patient based on the acquired tooth movement vector information to provide the orthodontic status information to a medical personnel account.

Preferably, the orthodontic image acquisition step includes: a process start step of starting a malocclusion confirmation process when the acquisition of the first tooth image is completed; a malocclusion classification step of, when the malocclusion confirming process starts, acquiring teeth arrangement state information about the patient by analyzing the first tooth image through the pre-stored algorithm to classify the acquired teeth arrangement state information as one of a plurality of malocclusion type information; and a solution information acquisition step of, when the teeth arrangement state information is classified as one of the plurality of malocclusion type information, acquiring treatment solution information about the classified malocclusion type information through a machine learning-based artificial intelligence solution generation algorithm that derives a solution for orthodontic treatment.

Preferably, in the orthodontic status information provision step, tooth movement vector information about each of the patient's teeth is acquired based on a common point included in a first cephalometric image corresponding to the first tooth part scan image and a second cephalometric image corresponding to the second tooth part scan data.

The common point may be a common location located on a patient's cephalic part included in the first cephalometric image and the second cephalometric image, in which the common point includes at least three locations that are not changed even when the patient's teeth arrangement is corrected, and may serve as a reference point for overlapping the first tooth image, the second tooth image, and the third tooth image.

The orthodontic status information provision step may include: an image overlapping step of generating a prognostic image by overlapping the first tooth image, the second tooth image, and the third tooth image based on the common point included in the first cephalometric image and the second cephalometric image; an orthodontic progress confirmation step of, when the generation of the prognostic image is completed, generating first tooth movement vector information including first tooth movement direction information and first tooth movement distance information based on the prognostic image by confirming a direction and distance in which each of the teeth is moved by comparing teeth arrangement corresponding to the first tooth image with teeth arrangement corresponding to the third tooth image; an orthodontic progress prediction step of, when a function of the orthodontic progress confirmation step is performed, generating second tooth movement vector information including second tooth movement direction information and second tooth movement distance information based on the prognostic image by confirming a direction and distance in which each of the teeth is expected to be moved by comparing the teeth arrangement corresponding to the first tooth image with teeth arrangement corresponding to the second tooth image; and an information generation step of, when the acquisition of the first tooth movement vector information and the second tooth movement vector information is completed, starting an information generation process by generating orthodontic status information based on the first tooth movement vector information and the second tooth movement vector information.

In the image overlapping step, the prognostic image may be generated by applying a graphic effect such that the first tooth image, the second tooth image, and the third tooth image, which overlap each other based on the common point, are visually distinguished.

The orthodontic progress prediction step may include: a fourth tooth image acquisition step of, when the direction and distance in which each of the teeth is expected to be moved, generating a plurality of fourth tooth images corresponding to each of a plurality of time points based on the treatment solution information from the first tooth image and the second tooth image; and a second tooth movement vector information generation step of, when the generation of the plurality of fourth tooth images is completed, generating the second tooth movement vector information about teeth arrangement included in each of the plurality of fourth tooth images by comparing each of the plurality of fourth tooth images in order of progress, and each of the plurality of time points may be at least two time points input by the medical personnel account.

The information generation step may include: a direction confirmation step of, when the generation of the first tooth movement vector information and the second tooth movement vector information is completed, confirming that an error rate of a movement axis direction based on the first tooth movement direction information with respect to a movement axis direction based on the second tooth movement direction information is equal to or less than a specified error rate by comparing the second tooth movement direction information with the first tooth movement direction information; a distance confirmation step of confirming that an error rate of a movement distance based on the first tooth movement distance information with respect to a movement distance based on the second tooth movement distance information is equal to or less than the specified error rate by comparing the second tooth movement distance information with the first tooth movement distance information during the direction confirmation step; and an orthodontic status information generation step of, when result information based on the direction confirmation step and the distance confirmation step is acquired, generating the orthodontic status information indicating a status of orthodontic treatment for the patient's teeth arrangement based on the result information.

Preferably, the method for providing an orthodontic status and orthodontic treatment evaluation information based on tooth part scan data of a patient further includes: an orthodontic treatment evaluation information provision step, wherein the orthodontic treatment evaluation information provision step includes: a target value acquisition step of, when a function of the initial image acquisition step is completed, acquiring a malocclusion image for patient's teeth based on the first tooth part scan data, acquiring treatment solution information based on the acquired malocclusion image, and acquiring an orthodontic target value for correcting the patient's malocclusion based on the acquired treatment solution information; an orthodontic completion image acquisition step of, in a state in which the acquisition of the orthodontic target value is completed, acquiring an orthodontic completion image that is an image for corrected teeth arrangement based on the third tooth part scan data when third tooth part scan data, which is new three-dimensional scan data acquired by capturing a patient who has completed the orthodontic treatment by the transparent orthodontic appliance; and an evaluation information provision step of acquiring an orthodontic achievement value for each of the corrected teeth based on the acquired orthodontic completion image, and when error information is acquired by comparing the orthodontic achievement value with the orthodontic target value, generating orthodontic treatment evaluation information, which is evaluation information about the orthodontic treatment, based on the acquired error rate to provide the orthodontic treatment evaluation information to the medical personnel account.

The target value acquisition step may include: a malocclusion confirmation start step of starting a malocclusion confirmation process when the first tooth part scan data is received from the medical personnel account; a malocclusion determination step of, when the malocclusion confirmation process is started, determining patient's malocclusion by analyzing the malocclusion image through a pre-stored malocclusion confirmation algorithm to confirm the patient's teeth arrangement through the analyzed malocclusion image, and classifying the confirmed teeth arrangement as any one of a plurality of malocclusion information; and a solution acquisition step of, when the determination on the patient's malocclusion is completed, acquiring treatment solution information about the patient's malocclusion through a machine learning-based artificial intelligence solution generation algorithm that derives a solution for orthodontic treatment.

In the malocclusion determination step, when the patient's teeth arrangement is confirmed, at least one of a position, a contact relationship between adjacent teeth, a vertical relationship, rotation, and inclination for each of the patient's teeth included in the malocclusion image may be confirmed through the pre-stored malocclusion confirmation algorithm.

The target value acquisition step may include: a guide application step of, when the acquisition of the treatment solution information is completed by performing a function of the solution acquisition step, applying an orthodontic guide based on the treatment solution information to the malocclusion image through the solution generation algorithm; a virtual orthodontic image acquisition step of, as the orthodontic guide based on the treatment solution information is applied to the malocclusion image, acquiring a virtual orthodontic image, which is a virtual image corresponding to teeth arrangement in which the orthodontic treatment of the patient's teeth is completed, by arranging each of the patient's teeth so as to be in a state in which the orthodontic treatment of the patient's teeth is completed; and an orthodontic value acquisition step of, when the acquisition of the virtual orthodontic image is completed, acquiring orthodontic target direction information and orthodontic target distance information about each of the teeth by comparing the virtual orthodontic image with the malocclusion image to acquire the orthodontic target value, which is a reference value for correcting the patient's malocclusion.

The evaluation information provision step may include: an achievement value acquisition step of, when the orthodontic completion image is acquired by performing a function of the orthodontic completion image acquisition step, acquiring the orthodontic achievement value for each of the corrected patient's teeth by analyzing the orthodontic completion image through the pre-stored malocclusion confirmation algorithm; an error value confirmation step of, when the acquisition of the orthodontic achievement value is completed, determining whether the acquired error value is within a range of a specified error value by comparing the orthodontic target value with the orthodontic achievement value to acquire an error value of the orthodontic achievement value for the orthodontic target value; and an error information analysis step of, based on a result determined according to a function of performing the error value confirmation step, generating the orthodontic treatment evaluation information by generating error information and analyzing the generated error information through the artificial intelligence solution generation algorithm.

The orthodontic treatment evaluation information may be information obtained by determining whether or not orthodontic treatment for each of the patient's teeth is successful based on the error information, and may include orthodontic improvement information for correcting each of the teeth that has failed the orthodontic treatment when it is determined that the orthodontic treatment for each of the patient's teeth based on the error information has failed, and the orthodontic improvement information may be information generated through the artificial intelligence solution generation algorithm, and may be exemplary treatment information derived based on orthodontic treatment history information about another patient learned through the artificial intelligence solution generation algorithm.

The evaluation information provision step may include: an orthodontic appliance design generation step of, when the orthodontic treatment evaluation information including the exemplary treatment information is provided to the medical personnel account, generating a first design, which is design information about the transparent orthodontic appliance based on the exemplary treatment information; and an orthodontic appliance design provision step of, when the generation of the first design is completed, providing an interface capable of comparing the first design with the second design to the medical personnel account by acquiring second design information, which is design information about the transparent orthodontic appliance based on the orthodontic target value.

When doctor opinion information for changing the design of the transparent orthodontic appliance is received from the medical personnel account, the interface may modify the first design based on the received doctor opinion information. According to one embodiment of the present invention, there is provided an apparatus for providing an orthodontic status based on tooth part scan data of a patient, which includes a computing device including one or more processors and one or more memories storing instructions executable by the processors, in which the apparatus includes: an initial image acquisition unit which acquires a first tooth image, which is an image for patient's teeth arrangement, based on received first tooth part scan data when first tooth part scan data, which is three-dimensional scan data acquired by capturing a patient's head, is received; an orthodontic image acquisition unit which, when the acquisition of the first tooth part image is completed, confirms a teeth arrangement state based on the first tooth image through a pre-stored algorithm, acquires treatment solution information for correcting the teeth arrangement based on the confirmed teeth arrangement state, and acquires a second tooth image, which is an image for predicted teeth arrangement upon orthodontic completion based on the acquired treatment solution information; an orthodontic appliance design generation unit which, when the acquisition of the second tooth image is completed, generates a design of a transparent orthodontic appliance for correcting the patient's teeth arrangement into teeth arrangement corresponding to the second tooth image; an intermediate image acquisition unit which acquires a third tooth image, which is an image for arrangement of patient's teeth being corrected, based on received second tooth part scan data when second tooth part scan data, which is new three-dimensional scan data, is received in a process of correcting the patient's teeth arrangement as the patient wears the transparent orthodontic appliance based on the generated design; and an orthodontic status information provision unit which, when the acquisition of the third tooth image is completed, acquires tooth movement vector information about the patient through the first tooth image, the second tooth image, and the third tooth image to generate orthodontic status information for orthodontic treatment of the patient based on the acquired tooth movement vector information to provide the orthodontic status information to a medical personnel account.

According to one embodiment of the present invention, there is provided a computer-readable recording medium that stores instructions for allowing a computing device to perform the following steps, wherein the steps include: an initial image acquisition step of acquiring a first tooth image, which is an image for patient's teeth arrangement, based on received first tooth part scan data when first tooth part scan data, which is three-dimensional scan data acquired by capturing a patient's head, is received; an orthodontic image acquisition step of, when the acquisition of the first tooth part image is completed, confirming a teeth arrangement state based on the first tooth image through a pre-stored algorithm, acquiring treatment solution information for correcting the teeth arrangement based on the confirmed teeth arrangement state, and acquiring a second tooth image, which is an image for predicted teeth arrangement upon orthodontic completion based on the acquired treatment solution information; an orthodontic appliance design generation step of, when the acquisition of the second tooth image is completed, generating a design of a transparent orthodontic appliance for correcting the patient's teeth arrangement into teeth arrangement corresponding to the second tooth image; an intermediate image acquisition step of acquiring a third tooth image, which is an image for arrangement of patient's teeth being corrected, based on received second tooth part scan data when second tooth part scan data, which is new three-dimensional scan data, is received in a process of correcting the patient's teeth arrangement as the patient wears the transparent orthodontic appliance based on the generated design; and an orthodontic status information provision step of, when the acquisition of the third tooth image is completed, acquiring tooth movement vector information about the patient through the first tooth image, the second tooth image, and the third tooth image, and generating orthodontic status information for orthodontic treatment of the patient based on the acquired tooth movement vector information to provide the orthodontic status information to a medical personnel account.

### Advantageous Effects

Through the method for providing an orthodontic status and orthodontic treatment evaluation information based on tooth part scan data of a patient according to the present invention, it is possible to determine a type of malocclusion for the patient's teeth arrangement, thereby providing the patient with a treatment plan suitable for the teeth arrangement state of the patient.

In addition, it is possible to rapidly analyze each of the patient's teeth through the pre-stored algorithm, thereby determining the type of malocclusion for the patient's teeth arrangement.

In addition, it is possible to correct the patient's teeth arrangement based on the common point, which is not changed due to the orthodontic treatment of the teeth arrangement in a cephalometric image based on the tooth part scan data of the patient, thereby correcting the teeth without aesthetic defects.

In addition, it is possible to analyze the arrangement of the patient's teeth being corrected through the artificial intelligence solution generation algorithm, thereby providing an orthodontic status information, which is status information about a teeth arrangement treatment in which the teeth are currently being corrected.

### DESCRIPTION OF DRAWINGS

FIG. 1 is a flowchart for explaining a method for providing an orthodontic status and orthodontic treatment evaluation information based on tooth part scan data of a patient according to one embodiment of the present invention.

FIG. 2 is a flowchart for explaining an orthodontic image acquisition step of the method for providing an orthodontic status and orthodontic treatment evaluation information based on tooth part scan data of a patient according to one embodiment of the present invention.

FIG. 3 is a view for explaining a pre-stored algorithm of the method for providing an orthodontic status and orthodontic treatment evaluation information based on tooth part scan data of a patient according to one embodiment of the present invention.

FIG. 4 is a view for explaining an orthodontic status information provision step of the method for providing an orthodontic status and orthodontic treatment evaluation information based on tooth part scan data of a patient according to one embodiment of the present invention.

FIG. 5 is a view for explaining an orthodontic progress confirmation unit of an apparatus for providing an orthodontic status based on tooth part scan data of a patient according to one embodiment of the present invention.

FIG. 6 is a view for explaining an orthodontic progress prediction step of the method for providing an orthodontic status and orthodontic treatment evaluation information based on tooth part scan data of a patient according to one embodiment of the present invention.

FIG. 7 is a view for explaining an information generation step of the method for providing an orthodontic status and orthodontic treatment evaluation information based on tooth part scan data of a patient according to one embodiment of the present invention.

FIG. 8 is a view for explaining an orthodontic treatment evaluation information provision unit of the apparatus for providing an orthodontic status and orthodontic treatment evaluation information based on tooth part scan data of a patient according to one embodiment of the present invention.

FIG. 9 is a block diagram for explaining a target value acquisition unit of the apparatus for providing an orthodontic status and orthodontic treatment evaluation information based on tooth part scan data of a patient according to one embodiment of the present invention.

FIG. 10 is a view for explaining a malocclusion determination unit of the apparatus for providing an orthodontic status and orthodontic treatment evaluation information based on tooth part scan data of a patient according to one embodiment of the present invention.

FIG. 11 is another block diagram for explaining the target value acquisition unit for the apparatus for providing an orthodontic status and orthodontic treatment evaluation information based on tooth part scan data of a patient according to one embodiment of the present invention.

FIG. 12 is a block diagram for explaining an evaluation information provision unit of the apparatus for providing an orthodontic status and orthodontic treatment evaluation information based on tooth part scan data of a patient according to one embodiment of the present invention.

FIG. 13 is a view for explaining an interface of the apparatus for providing an orthodontic status and orthodontic treatment evaluation information based on tooth part scan data of a patient according to one embodiment of the present invention.

FIG. 14 is a view showing one example of an internal configuration of a computing device according to one embodiment of the present invention.

### BEST MODE

Hereinafter, various embodiments and/or aspects will be disclosed with reference to drawings. In the following description, multiple concrete details will be disclosed in order to help general understanding of one or more aspects for the purpose of description. However, it will also be appreciated by those skilled in the art to which the present invention pertains that such aspect(s) may be practiced without the specific details. In the following description and accompanying drawings, specific exemplary aspects of one or more aspects will be described in detail. However, the aspects are exemplary, and some equivalents of various aspects may be used, and the descriptions herein are intended to include both the aspects and equivalents thereto.

It is not intended that any “embodiment”, “example”, “aspect”, “illustration”, and the like used in the specification is preferable or advantageous over any other “embodiment”, “example”, “aspect”, “illustration”, and the like.

Further, the terms “includes” and/or “including” mean that a corresponding feature/or component exists, but it should be appreciated that the terms “include” or “including” mean that presence or addition of one or more other features, components, and/or a group thereof is not excluded.

Further, terms including an ordinal number such as “first” or “second’ may be used for the names of various components, not limiting the components. The above terms are used merely for the purpose of distinguishing one element from another element. For example, a first component may be referred to as a second component and vice versa without departing the scope of the present invention. The term “and/or” includes a combination of a plurality of related enumerated items or any of the plurality of related enumerated items.

In addition, unless otherwise defined, all terms used herein, including technical or scientific terms, have the same meaning as commonly understood by one of ordinary skill in the art to which this invention belongs. Terms as those defined in generally used dictionaries are to be interpreted to have the meanings consistent with the contextual meanings in the relevant field of art, and are not to be interpreted to have idealistic or excessively formalistic meanings unless explicitly defined in the embodiments of the present invention.

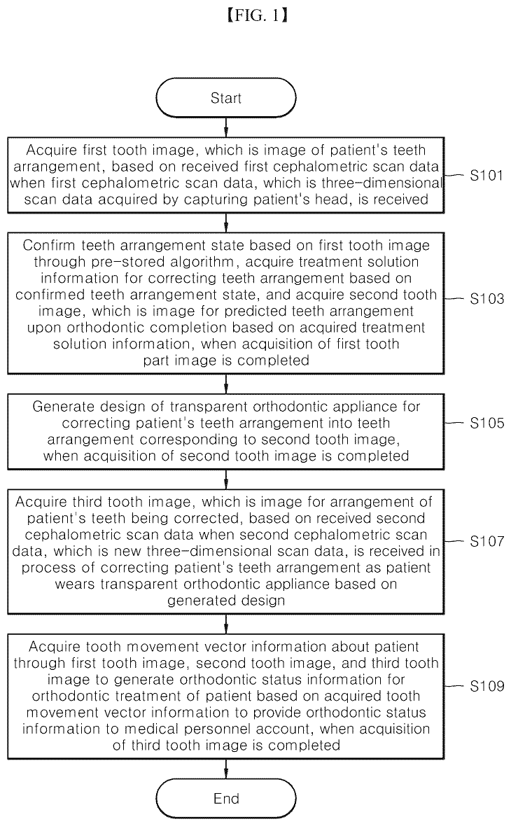

FIG. 1 is a flowchart for explaining a method for providing an orthodontic status and orthodontic treatment evaluation information based on tooth part scan data of a patient according to one embodiment of the present invention.

Referring to FIG. 1, a method for providing an orthodontic status and orthodontic treatment evaluation information based on tooth part scan data of a patient, which is implemented by a computing device including one or more processors and one or more memories storing instructions executable by the processors, may include an image acquisition step (S101), an orthodontic image acquisition step (S103), an orthodontic appliance design generation step (S105), an intermediate image acquisition step (S107), and an orthodontic status information provision step (S109).

In step S101, the one or more processors (hereinafter, referred to as a processor) may acquire a first tooth image, which is an image for patient's current teeth arrangement, based on received first tooth part scan data when first tooth part scan data, which is three-dimensional scan data acquired by capturing a patient's head, is received.

According to one embodiment, the first tooth part scan data may be a radiograph acquired by capturing the patient's head or an MRI image captured through an MRI device. That is, the tooth part scan data may be image data for patient's teeth arrangement included in the image acquired by capturing the patient's head. Further, the tooth part scan data may be image data acquired by capturing only the patient's teeth arrangement

According to one embodiment, when the first tooth part scan data is received, the processor may acquire a first tooth image, which is an image for the patient's current teeth arrangement, based on the tooth part scan data. The first tooth image may be an image for an initial state of the teeth arrangement, and the first tooth image may be an image acquired from the first tooth part scan data including a tooth image of the patient.

According to one embodiment, the processor may perform the orthodontic image acquisition step (S103) when the acquisition of the first tooth image is completed.

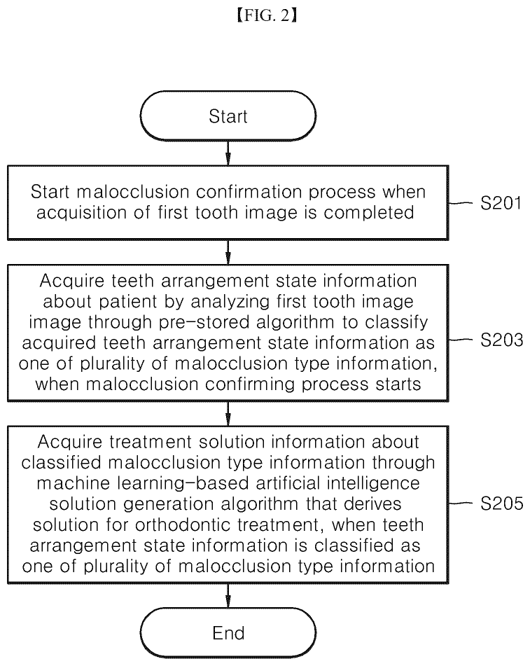

In step S103, when the acquisition of the first tooth image is completed, the processor may confirm the teeth arrangement state based on the first tooth image through a pre-stored algorithm, and may acquire treatment solution information for correcting the teeth arrangement based on the confirmed teeth arrangement state of the patient. In addition, the processor may acquire a second image that is an image for predicted tooth arrangement upon orthodontic completion based on the acquired treatment solution information. A detailed description of acquiring the treatment solution information will be described with reference to FIG. 2.

According to one embodiment, the processor may analyze the first tooth image through the pre-stored algorithm when the acquisition of the first tooth image is completed. The pre-stored algorithm may be a machine learning-based algorithm for analyzing the first tooth image to determine the shape and position of each tooth included in the first tooth image, confirming the teeth arrangement state of the patient according to the determination result, and classifying teeth arrangement state information corresponding to the confirmed teeth arrangement state as one of a plurality of malocclusion type information.

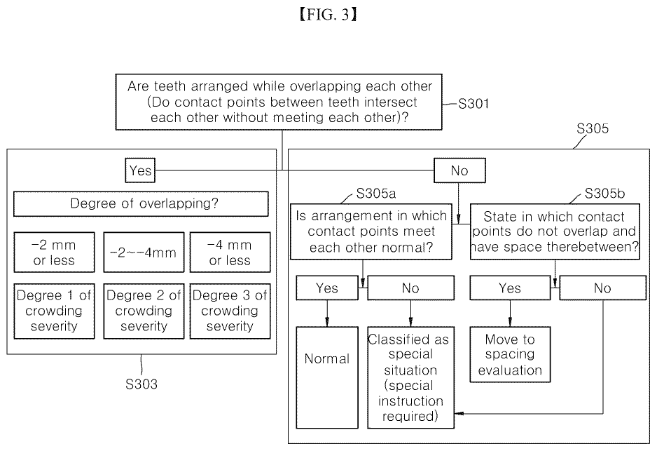

For example, the pre-stored algorithm may be an automatic recognition standardization algorithm. A detailed description of the automatic recognition standardization algorithm will be described with reference to FIGS. 2 and 3.

For another example, the pre-stored algorithm may be a PointNet-based deep learning algorithm. The processor may learn a previously acquired or input tooth image through the PointNet-based deep learning algorithm to determine the shape and position of each tooth (e.g., the first tooth, the second tooth, etc.) included in the tooth image to confirm the teeth arrangement state of the patient. The processor may be an algorithm for confirming the position of the mandible of the patient, the arrangement (shape and position) of the teeth, the relationship between the maxilla and the mandible, and the position and inclination of a maxillo-mandibular complex for the cranium through the first tooth image. That is, the pre-stored algorithm is not limited thereto as long as it is a machine learning-based algorithm capable of conforming the type of malocclusion of the patient by machine-learning the previously acquired or input tooth image as well as the PointNet-based deep learning algorithm to determine the teeth arrangement of the patient through a newly input tooth image.

According to one embodiment, when the analysis of the first tooth image is completed through the pre-stored algorithm, the processor may acquire treatment solution information using a machine learning-based artificial intelligence solution generation algorithm that derives a solution for correcting patient's malocclusion, based on teeth arrangement state information corresponding to the teeth arrangement state included in the analyzed first tooth image.

According to one embodiment, the processor may classify the confirmed teeth arrangement state of the patient as one of a plurality of malocclusion type information A detailed description of the plurality of malocclusion type information will be described with reference to FIG. 2. The processor may acquire the treatment solution information through the artificial intelligence solution generation algorithm based on the classified malocclusion type information. A detailed description of classifying the malocclusion type information will be described with reference to FIG. 3.

According to one embodiment, the artificial intelligence solution generation algorithm may be an algorithm for the processor to present visualized treatment objectives (VTOs) and an optimal treatment plan for patient's teeth arrangement by receiving data for orthodontic treatment from another electronic device (e.g., a desktop, a tablet PC, and a medical device) or a medical personnel account and machine-learning the received data.

According to one embodiment, the processor may machine-learn the received data as learning data when receiving the data for the orthodontic treatment and acquire visualized treatment objectives (VTOs) for the patient's teeth arrangement and information about an optimal treatment plan in consideration of the position of the stabilized mandibular warhead, the arrangement of the teeth having an appropriate angle, the relationship between the maxilla and mandible, and the position of inclination, etc. of the proper maxillo-mandibular complex for the cranium. That is, the treatment solution information may be information including at least one of VTO and treatment plan information generated by the artificial intelligence solution generation algorithm. Accordingly, the VTO and the treatment plan information include treatment method information, treatment period information, treatment drug information, and the like required for correcting the patient's teeth arrangement. Further, it will be apparent that the VTO is visualized treatment target information, and may include an image (e.g., a second tooth image) of a predicted tooth arrangement upon orthodontic completion of the patient based on the treatment plan information.

According to one embodiment, the second tooth image may be image information about the predicted tooth arrangement upon orthodontic completion, which is formed when the orthodontic treatment for malocclusion of the patient's teeth arrangement is completed according to the treatment plan information included in the treatment solution information. That is, the second tooth image may be an image generated by rearranging each of the teeth included in the first tooth image based on the treatment solution information through the solution generation algorithm.

According to one embodiment, the processor may perform the orthodontic appliance design generation step (S105) when the acquisition of the second tooth image is completed.

In step S105, when the acquisition of the second tooth image is completed, a design of a transparent orthodontic appliance may be generated in order to correct the patient's teeth arrangement into teeth arrangement corresponding to the second tooth image. The design may be design information required to manufacture the transparent orthodontic appliance by a 3D printer that is connected to the processor through wired/wireless network.

As the patient visits during treatment, the processor may perform the intermediate image acquisition step (S107) when new tooth part scan data is received.

In step S107, the processor may receive second tooth part scan data, which is new three-dimensional scan data, in a process of correcting the patient's teeth arrangement as the patient wears the transparent orthodontic appliance based on the generated design. That is, the second tooth part scan data is data acquired while the patient corrects the malocclusion, and may be data different from the first tooth part scan data including an image for patient's initial teeth arrangement.

According to one embodiment, when the second tooth part scan data is received, the processor may acquire a third tooth image, which is an image for arrangement of the patient's teeth being corrected, based on the received second tooth part scan data. The third tooth image may be an image for teeth arrangement of a patient whose malocclusion is being corrected by the transparent orthodontic appliance. The processor may acquire the third tooth image by analyzing the second tooth part scan data based on the pre-stored algorithm in order to acquire the third tooth image.

The processor may perform the orthodontic status information provision step (S109) when the acquisition of the third tooth image is completed.

In step S109, when the acquisition of the third tooth image is completed, the processor may generate tooth movement path information about the patient through the first tooth image, the second tooth image, and the third tooth image. The tooth movement path information may include movement direction information and movement distance information for each of the teeth to be moved, as the patient's teeth arrangement is corrected by the transparent orthodontic appliance. The processor may confirm an orthodontic status for the patient's teeth arrangement based on the tooth movement path information. For example, the processor may acquire oral state information after treatment for the teeth arrangement after the orthodontic treatment is completed by acquiring the tooth movement path information, based on the first tooth image and the third tooth image. In addition, the processor may acquire oral state information during treatment for arrangement of the teeth being corrected by acquiring the tooth movement path information, based on the second tooth image and the third tooth image.

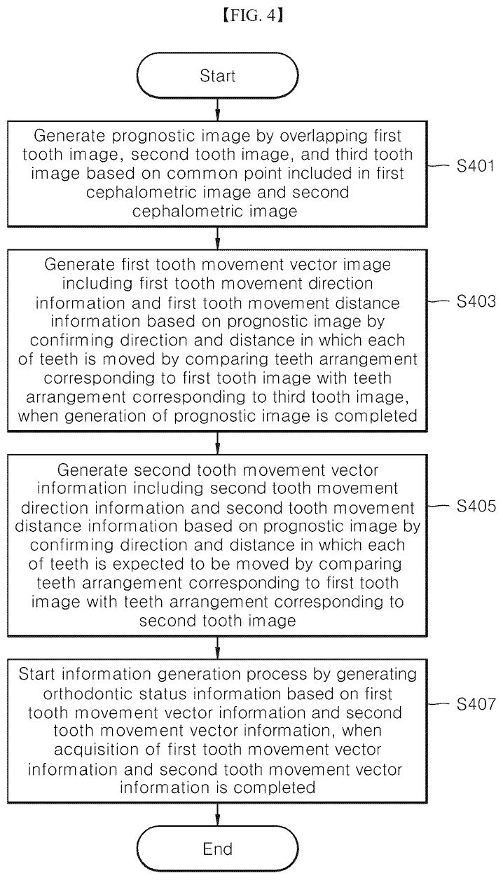

According to one embodiment, when the generation of the orthodontic status information is completed, the processor may provide orthodontic status corresponding to the generated orthodontic status information to the medical personnel account. A detailed description of generating the orthodontic status information will be described with reference to FIG. 4. In this case, the orthodontic status may mean information about a teeth arrangement state during the orthodontic treatment and information about a teeth arrangement state after completion of the orthodontic treatment.

FIG. 2 is a flowchart for explaining an orthodontic image acquisition step of the method for providing an orthodontic status and orthodontic treatment evaluation information based on tooth part scan data of a patient according to one embodiment of the present invention.

Referring to FIG. 2, the method for providing an orthodontic status and orthodontic treatment evaluation information based on tooth part scan data of a patient, which is implemented by a computing device including one or more processors and one or more memories storing instructions executable by the processors, may include an orthodontic image acquisition step (e.g., the orthodontic image acquisition step (S103) of FIG. 1).

According to one embodiment the one or more processors (hereinafter, referred to as a processor) may acquire a first tooth image, which is an image for patient's teeth arrangement, based on received first tooth part scan data when first tooth part scan data, which is three-dimensional scan data acquired by capturing a patient's head, is received. The first tooth part scan data may be input by the medical personnel account or received from an external device (e.g., desktop, tablet PC, medical devices).

According to one embodiment, the processor may perform the orthodontic image acquisition step when the acquisition of the first tooth image is completed. The orthodontic image acquisition step may include a process start step (S201), a malocclusion classification step (S203), and a solution information acquisition step (S205).

In step S201, the processor may start a malocclusion confirmation process when the acquisition of the first tooth image is completed. The malocclusion confirmation process may be a process for determining whether the teeth arrangement state of the patient is malocclusion.

According to one embodiment, when the malocclusion confirmation process is started, the processor may perform the malocclusion type confirmation step (S203).

In step S203, when the malocclusion confirmation process is started, the processor may acquire teeth arrangement state information about the patient by analyzing the first tooth image through the pre-stored algorithm. The teeth arrangement state information may include shape information and position information about each of the patient's teeth. The processor may classify the acquired teeth arrangement state information as one of a plurality of malocclusion type information. The plurality of malocclusion type information may be information serving as a reference required to classify which malocclusion the patient's teeth arrangement is among the plurality of malocclusions.

According to one embodiment, the pre-stored algorithm may be an automatic recognition standardization algorithm. The automatic recognition standardization algorithm may be a machine learning-based algorithm for classifying the teeth arrangement state information, which is acquired by confirming the shape and position of each of the patient's teeth included in the first tooth image by the processor, as one of the plurality of malocclusions. That is, the pre-stored algorithm may be an algorithm that provides treatment solution information when an algorithm learned based on a plurality of data (e.g., images of teeth of other patients) receives new three-dimensional scan data. That is, the processor may acquire the teeth arrangement state information through the pre-stored algorithm, may classify the information about the teeth arrangement state as malocclusion type information, and may provide treatment solution information based on the classified type of malocclusion.

The plurality of malocclusion type information may include at least one of crowding malocclusion, spacing malocclusion information, rotation malocclusion information, openbite & deepbite malocclusion information, mesiodistal tooth axis tilt (tipping) malocclusion information, buccolingual tooth axis tilt (torque) malocclusion information, and engagement malocclusion information. In addition, each of the plurality of malocclusion type information may include feature information about the patient's teeth arrangement derived from the first tooth image of the patient and the automatic recognition standardization algorithm, and history information until the teeth arrangement state information is classified as malocclusion information by the automatic recognition standardization algorithm. The method for classifying a type of malocclusion of the patient's teeth arrangement through the automatic recognition standardization algorithm will be described in detail with reference to FIG. 3.

According to one embodiment, the processor may perform the solution information acquisition step (S205) when the acquired teeth arrangement state information is classified as one of the plurality of malocclusion type information using the automatic recognition standardization algorithm, based on the first tooth image.

In step S205, when the teeth arrangement state information is classified as one of the plurality of malocclusion type information in step S203, the processor may acquire treatment solution information about the classified malocclusion type information through the machine learning-based artificial intelligence solution generation algorithm that derives a solution for orthodontic treatment.

For example, when the classified malocclusion type information is “crowding malocclusion type information”, the processor may generate treatment solution information by deriving the solution for correcting the crowding malocclusion corresponding to “crowding malocclusion type information” of the patient using the artificial intelligence solution generation algorithm. The processor may acquire treatment plan information about patient's malocclusion based on first tooth image information about the patient included in the crowding malocclusion type information. The processor may include, as the treatment plan information, treatment method information for causing at least one of teeth No. 11 and No. 21 not to be in contact with each other by changing a position of the at least one of teeth No. 11 and No. 21 when teeth No. 11 No. 21 corresponds to crowding malocclusion in the patient's teeth arrangement state.

That is, the processor may acquire treatment plan information and VTO information for correcting teeth arrangement corresponding to the first tooth image information through the first tooth image information and the artificial intelligence solution generation algorithm, and in this case, the acquired treatment plan information and VTO information may be information derived for the processor to correct the teeth arrangement corresponding to the first tooth image by learning a previously acquired tooth image through the artificial intelligence solution generation algorithm.

FIG. 3 is a view for explaining a pre-stored algorithm of the method for providing an orthodontic status and orthodontic treatment evaluation information based on tooth part scan data of a patient according to one embodiment of the present invention.

Referring to FIG. 3, the method for providing an orthodontic status and orthodontic treatment evaluation information based on tooth part scan data of a patient, which is implemented by a computing device including one or more processors and one or more memories storing instructions executable by the processors, may classify which type of malocclusion the patient's teeth arrangement is through the pre-stored algorithm (e.g., the automatic recognition standardization algorithm).

According to one embodiment, when the first tooth image is acquired, one or more processors (hereinafter, referred to as a processor) may analyze the first tooth image through the pre-stored algorithm (e.g., the automatic recognition standardization algorithm) to acquire teeth arrangement state information corresponding to a current teeth arrangement state of the patient. The teeth arrangement state information may include the shape and position of each tooth of the patient, as well as the position of the mandible of the patient, the relationship between the maxilla and the mandible, and the position and inclination information about the maxillo-mandibular complex for the cranium.

According to one embodiment, the processor may classify the acquired teeth arrangement state information as one of a plurality of malocclusions.

For example, the processor may acquire the teeth arrangement state information by confirming a state in which teeth No. 13 and No. 14 of the patient overlap each other based on the first tooth image. The processor may perform step S301 when the teeth arrangement state information is acquired. The processor may confirm whether teeth No. 13 and No. 14 are in contact with each other when step S301 is performed.

When it is confirmed that teeth No. 13 and No. 14 overlap each other while being in contact with each other, the processor may perform step S303. When it is confirmed that a degree of overlapping between teeth No. 13 and No. 14 is 3 mm based on the first tooth image, the processor may classify the patient's teeth arrangement state information as “crowding malocclusion information” by confirming the teeth arrangement state of the patient as “Degree 2 of crowding malocclusion”. The crowding malocclusion information may include first tooth image information about the patient, feature information (e.g., information about position relationship between teeth) about the patient's teeth arrangement derived by the automatic recognition standardization algorithm, and history information until the teeth arrangement state information is classified as malocclusion information by the automatic recognition standardization algorithm.

According to one embodiment, in the automatic recognition standardization algorithm, steps to be performed may be different according to the type of the malocclusion type information, and configurations (e.g., a distance between teeth, rotation of teeth, a vertical relationship between teeth, and an inclination for each tooth) required for performing the steps may be different. That is, FIG. 3 is a view showing a step for classifying the teeth arrangement state information about the patient as crowding malocclusion of the teeth in the automatic recognition standardization algorithm, and the corresponding steps may be different according to the malocclusion.

For another example, the processor may confirm a state in which teeth No. 13 and No. 14 are spaced apart from each other by identifying each of the patient's teeth based on the first tooth image. The processor may acquire teeth arrangement state information, which is information indicating an arrangement state in which teeth No. 13 and No. 14 are spaced apart from each other.

When the teeth arrangement state information is acquired, the processor may confirm a distance between teeth No. 13 and No. 14. When the distance between teeth No. 13 and No. 14 is equal to or shorter than 2 mm, the processor may confirm the arrangement state as “Degree 1 of spacing malocclusion”, thereby classifying the teeth arrangement state information as “spacing malocclusion information”. The spacing malocclusion information may include first tooth image information about the patient, feature information (e.g., information about position relationship between teeth) about the patient's teeth arrangement derived by the automatic recognition standardization algorithm, and history information until the teeth arrangement state information is classified as malocclusion information by the automatic recognition standardization algorithm.

FIG. 4 is a view for explaining an orthodontic status information provision step of the method for providing an orthodontic status and orthodontic treatment evaluation information based on tooth part scan data of a patient according to one embodiment of the present invention.

Referring to FIG. 4, the method for providing an orthodontic status and orthodontic treatment evaluation information based on tooth part scan data of a patient, which is implemented by a computing device including one or more processors and one or more memories storing instructions executable by the processors, may include an orthodontic status information provision step (e.g., the orthodontic status information provision step (S109) of FIG. 1).

According to one embodiment, the orthodontic status information provision step may include an image overlapping step (S401), an orthodontic progress confirmation step (S403), an orthodontic progress prediction step (S405), and an information generation step (S407).

According to one embodiment, when the orthodontic status information provision step is performed, one or more processors (hereinafter, referred to as a processor) may acquire tooth movement vector information about the patient through the first tooth image, the second tooth image, and the third tooth image to generate orthodontic status information about the orthodontic treatment of the patient based on the acquired tooth movement vector information and provide orthodontic status corresponding to the generated orthodontic status information to the medical personnel account. The orthodontic status may include at least one of information about an oral state (e.g., a teeth arrangement state) during the orthodontic treatment and information about an oral state (e.g., a tooth arrangement state) after completion of the orthodontic treatment. A detailed description related to the step performed before the orthodontic status information provision step is performed will be described with reference to FIGS. 1 to 3.

According to one embodiment, when the orthodontic status information provision step is performed, the processor may acquire tooth movement vector information about each of the patient's teeth based on a common point included in a first cephalometric image and a second cephalometric image. The first cephalometric image may be an image including the patient's cephalic part (head) that may be extracted from the first tooth part scan data. That is, the first tooth part scan data including the first cephalometric image may be image data including all the patient's head and tooth part. In addition, the second cephalometric image may be an image including the patient's cephalic part (head) that may be extracted from the second tooth part scan data. That is, the second tooth part scan data including the second cephalometric image may be image data including all the patient's head and tooth part.

A detailed description related to the tooth movement vector information will be described later.

In step S401, the processor may generate a prognosis image by overlapping the first tooth image, the second tooth image, and the third tooth image based on a common point included in the first cephalometric image and the second cephalometric image.

According to one embodiment, the first cephalometric image may be a radiological image for the patient's head corresponding to the first tooth part scan data (e.g., the first tooth part scan data of FIG. 1), and may be an image including the first tooth image and the second tooth image. According to one embodiment, the second cephalometric image may be a radiological image for the patient's head corresponding to the second tooth part scan data (e.g., the second tooth part scan data of FIG. 1), and may be an image including the third tooth image.

According to one embodiment, the common point may be a common location located on a patient's cephalic part included in the first cephalometric image and the second cephalometric image, in which the common point includes at least three locations that are not changed even when the patient's teeth arrangement is corrected. As the patient's teeth arrangement is corrected by the transparent orthodontic appliance, not only the position of the teeth simply changes, but also the shape of the patient's cranium may change due to a force of pushing and pulling each tooth by the transparent orthodontic appliance. When the shape of the patient's cranium is changed, there may be a problem in that the teeth arrangement corrected by the transparent orthodontic appliance is not corrected according to the treatment plan.

Accordingly, in the present invention, the patient's teeth arrangement may be corrected by the transparent orthodontic appliance, and the location included in the cranium, which is not changed by the pushing and pulling force of the transparent orthodontic appliance, may be set as the “common point,” and the cephalometric images of the patient may overlap each other based on the “common point,” thereby confirming only the alignment of the patient's teeth arrangement caused by the orthodontic treatment.

That is, the processor may generate a prognostic image, which is an image that enables confirmation of a change in the arrangement of each of the patient's teeth by overlapping the first tooth image and the second tooth image included in the first cephalometric image and the third tooth image included in the second cephalometric image, based on at least three common points included in the first cephalometric image and the second cephalometric image. That is, the prognostic image may be one image including the patient's teeth arrangement based on each tooth image in one image by overlapping the first tooth image, the second tooth image, and the third tooth image based on the common point.

According to one embodiment, the processor may perform the orthodontic progress confirmation step (S403) when the generation of the prognostic image is completed.

In step S403, when the generation of the prognostic image is completed, the processor may compare teeth arrangement corresponding to the first tooth image with teeth arrangement corresponding to the third tooth image based on the prognostic image. The processor may confirm a direction and distance in which each of the teeth is moved by comparing the teeth arrangements, and may extract first tooth movement vector information including first tooth movement direction information and first tooth movement distance information from the prognostic image to acquire the first tooth movement vector information.

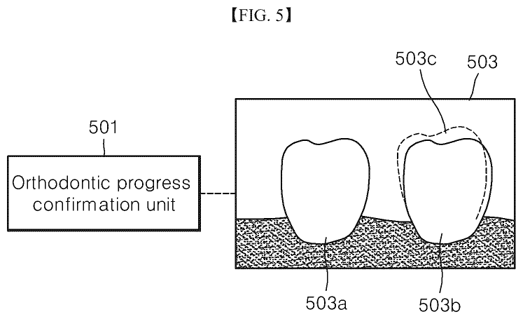

According to one embodiment, the first tooth image may be an initial image for the patient's teeth arrangement, and the third tooth image may be an intermediate image acquired during when the teeth arrangement of the patient is corrected through the transparent orthodontic appliance. That is, the processor may acquire substantial data about the orthodontic treatment of the teeth arrangement by confirming, through the position of each tooth included in the third image, the direction and distance in which each of the teeth included in the first tooth image is moved by the pushing and pulling force of the transparent orthodontic appliance. In this case, the processor may identify teeth included in each of the images (e.g., the first tooth image and the third tooth image) to confirm a vector value of the identified teeth. A detailed description of acquiring the first tooth movement vector information by the processor will be given with reference to FIG. 5.

In step S405, when a function of the orthodontic progress confirmation step is being performed, the processor may generate second tooth movement vector information including second tooth movement direction information and second tooth movement distance information based on the prognostic image by confirming a direction and distance in which each of the teeth is expected to be moved by comparing the teeth arrangement corresponding to the first tooth image with teeth arrangement corresponding to the second image.

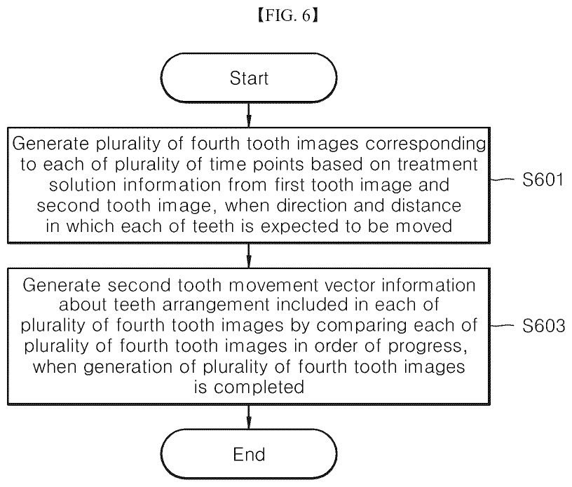

According to one embodiment, the first tooth image may be an initial image for the patient's teeth arrangement, and the second tooth image may be a predicted image in which orthodontic treatment of patient's malocclusion is completed based on the treatment solution information. A detailed description of generating the second tooth movement vector information by the processor will be given with reference to FIG. 6.

According to one embodiment, the processor may perform the orthodontic status information generation step (S407) when the generation of the second tooth movement vector information is completed.

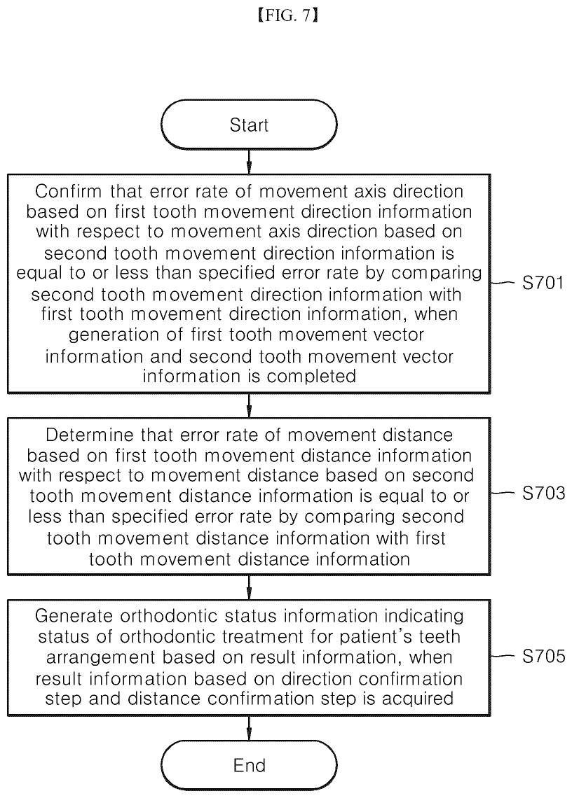

In step S407, when the acquisition of the first tooth movement vector information and the second tooth movement vector information is completed, the processor may start an information generation process of generating orthodontic treatment status information based on the first tooth movement vector information and the second tooth movement vector information. A detailed description of generating the orthodontic status information by the processor will be given with reference to FIG. 7.

FIG. 5 is a view for explaining an orthodontic progress confirmation unit of an apparatus for providing an orthodontic status based on tooth part scan data of a patient according to one embodiment of the present invention.

Referring to FIG. 5, the apparatus for providing an orthodontic status based on tooth part scan data of a patient, which is implemented by a computing device including one or more processors and one or more memories storing instructions executable by the processors, may include an orthodontic progress confirmation unit.

According to one embodiment, an orthodontic progress confirmation unit 501 may be a configuration of performing a function the same as a function performed in the orthodontic progress confirmation step shown in FIG. 4.

According to one embodiment, when the generation of a prognostic image 503 (e.g., the prognostic image of FIG. 4), the orthodontic progress confirmation unit 501 may compare teeth arrangements 503a and 503b corresponding to the first tooth image with teeth arrangements 503a and 503c corresponding to the third image. The teeth arrangements 503a and 503b for the first tooth image and the teeth arrangements 503a and 503c for the third tooth image may be images included in the prognostic image 503. The prognostic image may be an image to which a graphic effect is applied such that the first tooth image, the second tooth image, and the third tooth image, which overlap each other based on the common point of the first cephalometric image and the second cephalometric image, are visually distinguished.

According to one embodiment, the orthodontic progress confirmation step 501 may confirm a direction and distance in which each of the teeth is moved by comparing the teeth arrangements, and may extract first tooth movement vector information including first tooth movement direction information and first tooth movement distance information from the prognostic image 503 to acquire the first tooth movement vector information.

According to one embodiment, the first tooth image may be an initial image for the patient's teeth arrangement, and the third tooth image may be an intermediate image acquired during when the patient corrects the teeth arrangement through the transparent orthodontic appliance. That is, the orthodontic progress confirmation unit 501 may generate the first tooth movement vector information including information related to a direction and distance in which the teeth are substantially moved by the pushing and pulling force of the transparent orthodontic appliance by comparing the position of each tooth included in the first tooth image with the position of each tooth included in the third tooth image. In this case, the orthodontic progress confirmation unit 501 may identify the position of each tooth included in each image (e.g., the first tooth image and the third tooth image) to confirm a vector value of the position of the identified tooth.

For example, the orthodontic progress confirmation unit 501 may confirm the patient's teeth arrangement through the first tooth image. The orthodontic progress confirmation unit 501 may confirm that a space between teeth No. 13 and No. 14 is formed through the first tooth image. A detailed description of confirming the patient's teeth arrangement through the tooth image will be given with reference to the description of the automatic recognition standardization algorithm of FIG. 2.

In addition, the orthodontic progress confirmation unit 501 may confirm the patient's teeth arrangement through the third tooth image. The orthodontic progress confirmation unit 501 may confirm that tooth No. 14 503c is corrected in a direction in which tooth No. 13 503a is located by a predetermined distance through the third tooth image. In this case, the orthodontic progress confirmation unit may compare a vector value of tooth No. 14 503b of the first tooth image with a vector value of tooth No. 14 503c of the third tooth image to confirm a vector value of tooth No. 14 being corrected by the transparent orthodontic appliance.

The orthodontic treatment progress confirmation unit 501 may confirm the vector value for tooth No. 14 to generate the first tooth movement vector information including the first tooth movement distance information based on the distance in which tooth No. 14 is moved and the first tooth movement direction information based on the direction in which tooth No. 14 is moved.

FIG. 6 is a view for explaining an orthodontic progress prediction step of the method for providing an orthodontic status and orthodontic treatment evaluation information based on tooth part scan data of a patient according to one embodiment of the present invention.

Referring to FIG. 6, the method for providing an orthodontic status and orthodontic treatment evaluation information based on tooth part scan data of a patient, which is implemented by a computing device including one or more processors and one or more memories storing instructions executable by the processors, may include an orthodontic progress prediction step (e.g., the orthodontic progress prediction step (S405) of FIG. 4). The orthodontic progress prediction step may include a fourth tooth image acquisition step (S601) and a second tooth movement vector information generation step (S603).

According to one embodiment, one or more processors (hereinafter, referred to as a processor) may perform the orthodontic progress prediction step when the orthodontic progress confirmation step shown in FIG. 4 is being performed.

In step S601, when confirming the direction and distance in which each of the teeth is expected to be moved, the processor may acquire a plurality of fourth tooth images corresponding to each of a plurality of time points based on the treatment solution information from the first tooth image and the second tooth image.

More specifically, the processor may generate a plurality of fourth tooth images corresponding to each of the plurality of time points, based on the second tooth image in which each of the teeth in the first tooth image is rearranged (arrangement in which orthodontic treatment of the teeth is completed) based on the treatment solution information. Each of the plurality of time points may be at least two time points input by the medical personnel account. That is, the processor may acquire a plurality of images based on a process of correcting the teeth arrangement corresponding to the first tooth image into teeth arrangement corresponding to the second tooth image, and may acquire a fourth tooth image that is an image corresponding to each of the plurality of time points input by the medical personnel account among the plurality of acquired images.

According to one embodiment, the processor may acquire the second tooth image for predicted teeth arrangement upon orthodontic completion, based on the treatment solution information about the malocclusion type information about the patient generated by performing the orthodontic image acquisition step (e.g., the orthodontic image acquisition step (S103)). In this case, the acquired second tooth image may be an image for arrangement of the corrected teeth as the teeth arrangement based on the first tooth image is rearranged by the artificial intelligence solution generation algorithm.

Accordingly, the processor acquires a plurality of images including teeth arrangement based on a process of correcting the teeth arrangement corresponding to the first tooth image into the teeth arrangement corresponding to the second tooth image. In this case, the plurality of images acquired at this time may be acquired in order of the progress of the arrangement of corrected teeth. That is, the plurality of fourth teeth images may be images corresponding to at least two or more time points input by the medical personnel account among images based on the patient's teeth arrangement to be corrected based on the treatment solution information.

According to one embodiment, the processor may perform the second tooth movement vector information generation step (S603) when the acquisition of the plurality of fourth tooth images is completed.

In step S603, the processor may generate the second tooth movement vector information about teeth arrangement included in each of the plurality of fourth tooth images by comparing each of the plurality of fourth tooth images in order of progress, when the generation of the plurality of fourth tooth images is completed. The second tooth movement vector information may include second tooth movement path information and second tooth movement direction information. In this case, the second tooth movement vector information may acquire a larger number of fourth tooth images, which are images corresponding to the plurality of time points, as the plurality of time points input by the medical personnel account increases. That is, at least one or more second tooth movement vector information may be generated according to the number of the fourth tooth images, and this is because the teeth arrangements included in the plurality of fourth tooth images are different from each other, and thus the second tooth movement vector is generated by confirming the different teeth arrangements.

According to one embodiment, the second tooth movement direction information may be information indicating a movement distance in which each of the teeth is moved when the patient's teeth arrangement is corrected based on the treatment solution information. The second tooth movement distance information may be information indicating a movement direction in which each of the teeth is moved when the patient's teeth arrangement is corrected based on the treatment solution information.

More specifically, the processor may confirm the position of the teeth included in each of the plurality of fourth tooth images generated based on the first tooth image and the second tooth image. The processor may generate the second tooth movement vector information related to a distance and direction in which the position of the teeth included in each of the plurality of fourth tooth images is moved. by comparing each of the plurality of fourth tooth images in order of progress.

FIG. 7 is a view for explaining an information generation step of the method for providing an orthodontic status and orthodontic treatment evaluation information based on tooth part scan data of a patient according to one embodiment of the present invention.

Referring to FIG. 7, the method for providing an orthodontic status and orthodontic treatment evaluation information based on tooth part scan data of a patient, which is implemented by a computing device including one or more processors and one or more memories storing instructions executable by the processors, may include an information generation step (e.g., the information generation step (S407) of FIG. 4). The information generation step may include a direction confirmation step (S701), a distance confirmation step (S703), and an orthodontic status information generation step (S705).

According to one embodiment, one or more processors (hereinafter, referred to as a processor) may perform the direction confirmation step (S701) when the generation of the first tooth movement vector information and the second tooth movement vector information shown in FIG. 4 is completed.

In step S701, when the generation of the first tooth movement vector information and the second tooth movement vector information is completed, the processor may compare the second tooth movement direction information with the first tooth movement direction information. The second tooth movement direction information may be information included in the second tooth movement vector information. The first tooth movement direction information may be information included in the first tooth movement vector information.

According to one embodiment, the processor may confirm whether an error rate of a movement axis direction (e.g., a first movement axis direction) based on the first tooth movement direction information with respect to a movement axis direction (e.g., a second movement axis direction) based on the second tooth movement direction information is equal to or less than a specified error rate. The specified error rate mentioned in the direction confirmation step may mean a coordinate error rate. The movement axis direction may mean a direction in which each of the patient's teeth is to be moved based on the treatment solution information. The specified error rate may be a numerical value serving as a reference for generating the orthodontic status information.

According to one embodiment, the processor may compare x, y, and z values based on the first movement axis direction with x, y, and z values based on the second movement axis direction. The processor may acquire error values of the x, y, and z values based on the first movement axis direction with respect to the x, y, and z values based on the second movement axis direction, and may acquire an absolute value of the acquired error values. The processor may acquire an average value of the acquired absolute value, in which the average value may be an error rate of the first tooth movement direction information with respect to the second tooth movement direction information. The processor may confirm whether the acquired error rate of the movement direction information is equal to or less than the specified error rate.

According to one embodiment, when the direction confirmation step is being performed, the processor may perform the distance confirmation step (S703).

In step S703, the processor may compare the second tooth movement distance information with the first tooth movement distance information when the direction confirmation step is being performed. The second tooth movement distance information may be information included in the second tooth movement vector information. The first tooth movement distance information may be information included in the first tooth movement vector information.

According to one embodiment, the processor may confirm whether an error rate of a movement distance (e.g., a first movement distance) based on the first tooth movement distance information with respect to a movement distance (e.g., a second movement distance) based on the second tooth movement distance information is equal to or less than a specified error rate. The specified error rate mentioned in the distance confirmation step may mean a specified distance error rate. The movement distance may mean a distance in which each of the patient's teeth is to be moved based on the treatment solution information.

According to one embodiment, the processor may compare a movement distance value based on a first movement distance of a specific tooth with a movement distance value based on a second movement distance of the specific tooth. In this case, the processor may acquire an error value of the first movement distance with respect to the second movement distance. The acquired error value may be an error rate for the movement distance. The processor may confirm whether the acquired error rate of the movement distance is equal to or less than a specified error rate.

According to one embodiment, when the direction confirmation step (S701) and the distance confirmation step (S703) are completed, the processor may perform the orthodontic status information generation step (S705).

In step S705, the processor may acquire result information based on the direction confirmation step (S701) and the distance confirmation step (S703). The result information may be information including a result of confirming whether the error rate for the movement direction is equal to or less than the specified error rate and a result of confirming whether the error rate for the movement distance is equal to or less than the specified error rate.

For example, when the error rate for the movement direction is 4 and the specified error rate for the movement direction is 5, the processor may determine that the error rate for the movement direction is equal to or less than the specified error rate. In addition, when the error rate for the movement distance is 4 and the specified error rate for the movement distance is 7, the processor may determine that the error rate for the movement distance is equal to or less than the specified error rate, thereby acquiring result information including the determination results. The result information may include at least one of text information, image information, and video information based on the determination results.

According to one embodiment, when the acquisition of the result information is completed, the processor may generate orthodontic status information indicating a status of orthodontic treatment for the patient's teeth arrangement based on the treatment solution information and the result information.

More specifically, the processor may analyze a cause of the error rate based on the result information through an artificial intelligence solution generation algorithm (e.g., the artificial intelligence solution generation algorithm of FIG. 2). Since the artificial intelligence solution generation algorithm learns various data (a tooth image of another patient and history data acquired during orthodontic treatment of another patient), it is possible to derive cause information in which an error rate has occurred based on the result information. In this case, the processor may analyze each of the teeth having an error rate equal to or greater than the specified error rate when analyzing the result information through the artificial intelligence solution generation algorithm, and may generate orthodontic status information indicating a teeth arrangement state of the patient by each of the analyzed teeth.

In addition, the processor may analyze the result information through the artificial intelligence solution generation algorithm to derive element information that may affect the arrangement of the teeth being corrected. The information derived as described above may be generated as orthodontic status information indicating a status for orthodontic treatment of the patient's teeth arrangement.

According to one embodiment, the processor may analyze the result information through the artificial intelligence solution generation algorithm. The processor may acquire new treatment solution information when the patient's teeth arrangement based on the result information analyzed through the artificial intelligence solution generation algorithm is not corrected to correspond to the treatment solution information. The processor may include the generated new treatment solution information in the orthodontic status information.

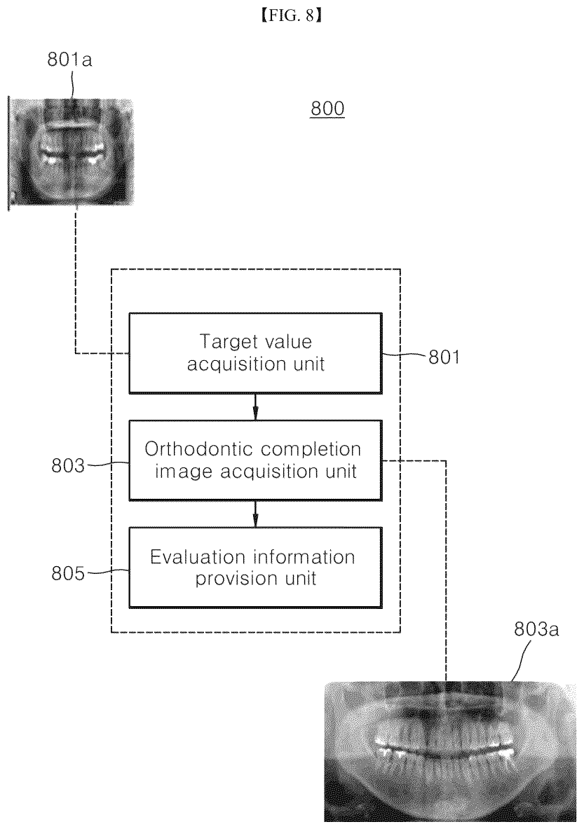

FIG. 8 is a view for explaining an orthodontic treatment evaluation information provision unit of the apparatus for providing an orthodontic status and orthodontic treatment evaluation information based on tooth part scan data of a patient according to one embodiment of the present invention.

Referring to FIG. 8, an apparatus 800 for providing an orthodontic status and orthodontic treatment evaluation information based on tooth part scan data (hereinafter, referred to as an apparatus for providing an orthodontic status and treatment evaluation information), which is implemented by a computing device including one or more processors and one or more memories storing instructions executable by the processors, may include a target value acquisition unit 801, an orthodontic completion image acquisition unit 803, and an evaluation information provision unit 805.

According to one embodiment, the target value acquisition unit 801 may receive first tooth part scan data 801a, which is three-dimensional scan data acquired by capturing the patient, from the medical personnel account. The first tooth part scan data 801a may be a radiological image acquired by capturing the patient. For example, the first tooth part scan data 801a may be image data acquired by capturing the patient's head. In this case, the acquired image data may be data including an image for the patient's head and teeth arrangement. In addition, the first tooth part scan data 801a may be image data acquired by capturing only the tooth part of the patient, that is, the patient's teeth arrangement.

According to one embodiment, the target value acquisition unit 801 may acquire a malocclusion image for the patient's teeth based on the received first tooth part scan data 801a. The malocclusion image is an image for patient's teeth arrangement before orthodontic treatment, which is acquired based on the first tooth part scan data 801a, and is an initial image including an image for patient's malocclusion.

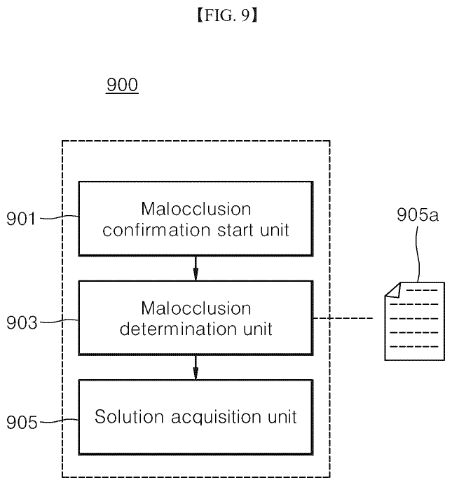

According to one embodiment, the target value acquisition unit 801 may acquire treatment solution information based on the acquired malocclusion image. The treatment solution information may be treatment information for correcting the patient's malocclusion based on the malocclusion image. A detailed description of acquiring the treatment solution information will be described with reference to FIG. 9.

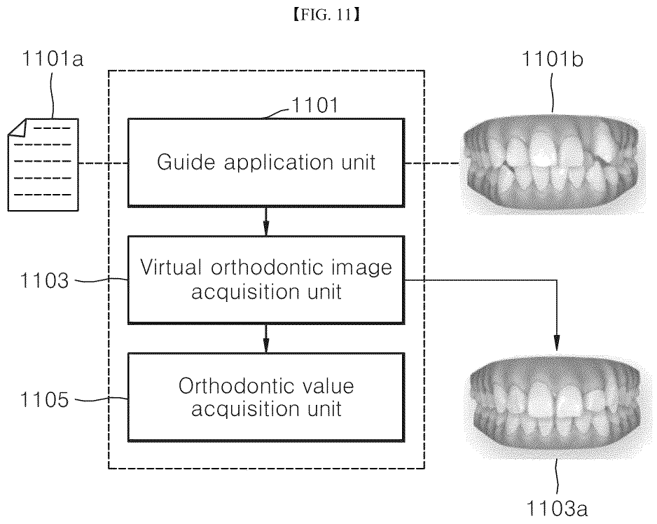

According to one embodiment, when the acquisition of the treatment solution information is completed, the target value acquisition unit 801 may acquire an orthodontic target value for correcting the patient's malocclusion based on the acquired treatment solution information. In this case, in order to correct the patient's malocclusion included in the malocclusion image into normal teeth arrangement, the target value acquisition unit 801 may acquire orthodontic target direction information and orthodontic target distance information for each of the patient's teeth corresponding to the malocclusion image based on the treatment solution information, and may acquire an orthodontic target value based on the acquired orthodontic target direction information and orthodontic target distance information. A detailed description of acquiring the orthodontic target value will be described with reference to FIG. 11.

According to one embodiment, the orthodontic completion image acquisition unit 803 may acquire third tooth part scan data, which is new three-dimensional scan data acquired by capturing a patient whose orthodontic treatment is completed by the transparent orthodontic appliance, in a state in which the acquisition of the orthodontic target value is completed by the target value acquisition unit 801. The third tooth part scan data may be a radiological image acquired by capturing the patient at a time point when the orthodontic treatment for the patient's malocclusion is completed.

According to one embodiment, when the reception of the third tooth part scan data is completed, the orthodontic completion image acquisition unit 803 may acquire an orthodontic completion image 803a, which is an image for arrangement of the corrected teeth, based on the third tooth part scan data. The orthodontic completion image may be an image for teeth arrangement of a patient whose orthodontic treatment is completed by the transparent orthodontic appliance.

According to one embodiment, when the acquisition of the orthodontic completion image 803 is completed, the evaluation information provision unit 805 may acquire an orthodontic achievement value for each of the corrected teeth based on the orthodontic completion image. The orthodontic achievement value may be information generated based on orthodontic distance achievement information and orthodontic direction achievement information for each of the teeth that is substantially corrected by the transparent orthodontic appliance.

According to one embodiment, when the acquisition of the orthodontic achievement value is completed, the evaluation information provision unit 805 may acquire error information about the orthodontic achievement value for the orthodontic achievement value by comparing the orthodontic achievement value with the orthodontic target value. The error information may be information generated according to whether the orthodontic achievement value satisfies the orthodontic target value. The evaluation information provision unit 805 may generate orthodontic treatment evaluation information, which is evaluation information about the orthodontic treatment, based on the error information to provide the orthodontic treatment evaluation information to the medical personnel account. The orthodontic treatment evaluation information is information generated based on the error information, and may be information for determining whether the orthodontic treatment of the patient is inadequate or effective.

FIG. 9 is a block diagram for explaining a target value acquisition unit of the apparatus for providing an orthodontic status and orthodontic treatment evaluation information based on tooth part scan data of a patient according to one embodiment of the present invention.

Referring to FIG. 9, the apparatus for providing an orthodontic status and orthodontic treatment evaluation information based on tooth part scan data (e.g., the apparatus 800 for providing an orthodontic status and treatment evaluation information based on tooth scan data of FIG. 8) (hereinafter, referred to as an apparatus for providing an orthodontic status and treatment evaluation information), which is implemented by a computing device including one or more processors and one or more memories storing instructions executable by the processors, may include a target value acquisition unit 900 (e.g., the target value acquisition unit (801) of FIG. 8).

According to one embodiment, the target value acquisition unit 900 may include a malocclusion confirmation start unit 901, a malocclusion determination unit 903, and a solution acquisition unit 905.

According to one embodiment, the malocclusion confirmation start unit 901 may start a malocclusion confirmation process when the first tooth part scan data, which is three-dimensional scan data acquired by capturing the patient, is received from the medical personnel account. The malocclusion confirmation process may be a process for determining patient's malocclusion by classifying which malocclusion information the patient's teeth arrangement is among a plurality of malocclusion information by confirming the patient's teeth arrangement through the malocclusion image.

According to one embodiment, the malocclusion determination unit 903 may confirm a teeth arrangement state of the patient by determining the shape and position of each tooth included in the malocclusion image through a pre-stored malocclusion confirmation algorithm. The malocclusion determination unit 903 may classify the patient's teeth arrangement as any one of the plurality of malocclusion information based on the confirmed teeth arrangement state. More specifically, when the teeth arrangement state of the patient is confirmed by analyzing the malocclusion image through the pre-stored orthodontic confirmation algorithm, the orthodontic determination unit 903 may classify the teeth arrangement state information corresponding to the confirmed teeth arrangement state as any one of the plurality of malocclusion information.

According to one embodiment, the pre-stored malocclusion algorithm may be a machine learning-based algorithm for determining the shape and position of each tooth included in the malocclusion image by analyzing the malocclusion image. For example, the pre-stored malocclusion confirmation algorithm may be a PointNet-based deep learning algorithm. The malocclusion determination unit 903 may learn previously acquired or input tooth images through the PointNet-based deep learning algorithm to determine the shape and position of each tooth (e.g., the first tooth, the second tooth, etc.) included in the tooth images to confirm the teeth arrangement state of the patient. In addition, the malocclusion determination unit may be an algorithm for confirming the position of the mandible of the patient, the arrangement (shape and position) of the teeth, the relationship between the maxilla and the mandible, and the position and inclination of the maxilla-mandibular complex for the cranium through the malocclusion image by using the pre-stored malocclusion confirmation algorithm.

That is, the pre-stored malocclusion confirmation algorithm is not limited thereto as long as it is a machine learning-based algorithm capable of conforming the type of malocclusion of the patient by machine-learning the previously acquired or input tooth image as well as the PointNet-based deep learning algorithm to determine the teeth arrangement of the patient through a newly input tooth image.

For another example, the pre-stored malocclusion confirmation algorithm may be an automatic recognition standardization algorithm. The automatic recognition standardization algorithm may be a machine learning-based algorithm for classifying the teeth arrangement state information, which is acquired by confirming the shape and position of each of the patient's teeth included in the malocclusion image by the malocclusion determination unit 903, as one of the plurality of malocclusion information.

According to one embodiment, when the patient's teeth arrangement is confirmed by analyzing the malocclusion image through the pre-stored malocclusion confirmation algorithm, the malocclusion determination unit 903 may confirm at least one of a position, a contact relationship between adjacent teeth, a vertical relationship, rotation, and inclination for each of the patient's teeth included in the malocclusion image through the pre-stored malocclusion confirmation algorithm. When the confirmation of the patient's teeth arrangement is completed, the malocclusion determination unit 903 may acquire teeth arrangement state information corresponding to the confirmed patient's teeth arrangement through the pre-stored malocclusion confirmation algorithm. That is, the teeth arrangement state information may include position information about each of the patient's teeth of the patient, contact relationship information with adjacent teeth, vertical relationship information with adjacent teeth, rotation information, and inclination information based on the malocclusion image. A detailed method for classifying the teeth arrangement state information as any one of the plurality of malocclusion information will be described with reference to FIG. 3.

According to one embodiment, when the teeth state information is classified as any one of the plurality of malocclusion information, the malocclusion determination unit 903 may determine the malocclusion corresponding to the classified malocclusion information as malocclusion for the patient's teeth arrangement.

According to one embodiment, when the determination of the patient's malocclusion by the malocclusion determination unit 903 is completed, the solution acquisition unit 905 may acquire treatment solution information about the patient's malocclusion through the machine learning-based artificial intelligence solution generation algorithm that derives a solution for the orthodontic treatment. Since the artificial intelligence solution generation algorithm learns various data (a tooth image of another patient, history data acquired during orthodontic treatment of another patient), it is possible to derive cause information in which an error rate has occurred based on the result information.

According to one embodiment, the artificial intelligence solution generation algorithm may be an algorithm for the solution acquisition unit 905 to present visualized treatment objectives (VTOs) and an optimal treatment plan for patient's teeth arrangement by receiving data for orthodontic treatment from another electronic device (e.g., a desktop, a tablet PC, and a medical device) or a medical personnel account and machine-learning the received data.

According to one embodiment, the solution acquisition unit 905 may machine-learn the received data as learning data when receiving tooth image data about another patient, and may acquire visualized treatment objectives (VTOs) for the patient's teeth arrangement and information about an optimal treatment plan in consideration of the position of the stabilized mandibular warhead, the arrangement of the teeth having an appropriate angle, the relationship between the maxilla and the mandible, and the position of inclination, etc. of the proper maxillo-mandibular complex for the cranium.

That is, the treatment solution information may be information including at least one of VTO and treatment plan information generated by the artificial intelligence solution generation algorithm. Accordingly, the VTO and the treatment plan information include treatment method information, treatment period information, treatment drug information, and the like required for correcting the patient's teeth arrangement.

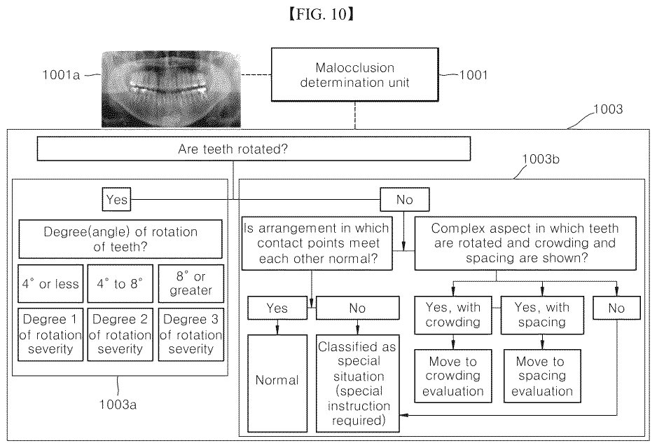

FIG. 10 is a view for explaining a malocclusion determination unit of the apparatus for providing an orthodontic status and orthodontic treatment evaluation information based on tooth part scan data of a patient according to one embodiment of the present invention.

Referring to FIG. 10, when the malocclusion confirmation process is started, a malocclusion determination unit 1001 (e.g., the malocclusion determination unit 903 of FIG. 9) may confirm the patient's teeth arrangement through a received malocclusion image 901a.

According to one embodiment, the malocclusion determination unit 1001 may confirm a teeth arrangement state of the patient by determining the shape and position of each of the teeth included in a malocclusion image 1001a through a pre-stored malocclusion confirmation algorithm. A detailed description of confirming the patient's teeth arrangement through the pre-stored malocclusion confirmation algorithm by the malocclusion determination unit 1001 will be given with reference to FIG. 9.

According to one embodiment, the malocclusion determination unit 1001 may classify the patient's teeth arrangement as any one of the plurality of malocclusion information based on the confirmed teeth arrangement state. In this case, the malocclusion determination unit 1001 may classify the patient's teeth arrangement as any one of the plurality of malocclusion information based on the acquired teeth arrangement state information. The plurality of malocclusion type information may include at least one of crowding malocclusion, spacing malocclusion information, rotation malocclusion information, openbite & deepbite malocclusion information, mesiodistal tooth axis tilt (tipping) malocclusion information, buccolingual tooth axis tilt (torque) malocclusion information, and engagement malocclusion information.

According to one embodiment, when the malocclusion image is acquired, the malocclusion determination unit 1001 may analyze the malocclusion image through the pre-stored malocclusion confirmation algorithm (e.g., the automatic recognition standardization algorithm) to acquire teeth arrangement state information corresponding to the teeth arrangement state of the patient. The teeth arrangement state information may include the shape and position of each tooth of the patient, as well as the position of the mandible of the patient, the relationship between the maxilla and the mandible, and the position and inclination information about the maxillo-mandibular complex for the cranium.

For example, when the teeth arrangement state information is acquired, the malocclusion determination unit 1001 may start a determination process 1003 for classifying the teeth arrangement state information as one of the plurality of malocclusion information. The malocclusion determination unit 1001 may confirm a state in which tooth No. 13 of the patient is rotated based on the teeth arrangement state information. When tooth No. 13 is rotated, the malocclusion determination unit 1001 may perform a process included in 1003a. In addition, when it is confirmed that the tooth of the patient is not rotated based on the teeth arrangement state information, the malocclusion determination unit 1001 may perform a process included in 1003b. The determination process 1003 may be a different process for each of the plurality of types of malocclusion.

FIG. 11 is another block diagram for explaining the target value acquisition unit for the apparatus for providing an orthodontic status and orthodontic treatment evaluation information based on tooth part scan data of a patient according to one embodiment of the present invention.

Referring to FIG. 11, the apparatus for providing an orthodontic status and orthodontic treatment evaluation information based on tooth part scan data (e.g., the apparatus 800 for providing an orthodontic status and treatment evaluation information based on tooth scan data of FIG. 8) (hereinafter, referred to as an apparatus for providing an orthodontic status and treatment evaluation information), which is implemented by a computing device including one or more processors and one or more memories storing instructions executable by the processors, may include a target value acquisition unit 1100 (e.g., the target value acquisition unit (801) of FIG. 8).

According to one embodiment, the target value acquisition unit 1100 may include a guide application unit 1101, a virtual orthodontic image acquisition unit 1103, and an orthodontic value acquisition unit 1105.

According to one embodiment, when the acquisition of treatment solution information 1101a by a solution acquisition unit (e.g., the solution acquisition unit 905 of FIG. 9) is completed, the target value acquisition unit 1101 may apply an orthodontic guide based on the treatment solution information 1101a to a malocclusion image 1101b of the patient through a solution generation algorithm. A detailed description related to the solution generation algorithm will be described with reference to FIG. 9. The orthodontic guide is information applied to the orthodontic image 1101b representing patient's initial teeth arrangement, and may be vector information about each of the teeth for arranging the patient's malocclusion based on the malocclusion image 1101b into teeth arrangement in which orthodontic treatment is completed in an optimal form based on the treatment solution information 1101a.

According to one embodiment, as the orthodontic guide based on the treatment solution information 1101a is applied to the malocclusion image 1101b, the virtual orthodontic image acquisition unit 1103 may virtually arrange each of the patient's teeth to be in a state in which orthodontic treatment of the teeth is completed, thereby acquiring a virtual orthodontic image 1103a, which is a virtual image corresponding to the corrected teeth arrangement.

According to one embodiment, the virtual orthodontic image acquisition unit 1103 may acquire the virtual orthodontic image 1103a corresponding to teeth arrangement in which the orthodontic treatment of the patient is completed based on the treatment solution information 1101a by changing the vector information about each of the teeth included in the malocclusion image 1101b based on the vector information about each of the teeth based on the orthodontic guide.

According to one embodiment, when the acquisition of the virtual orthodontic image 1103a is completed, the orthodontic value acquisition unit 1105 may compare the virtual orthodontic image 1103a with the malocclusion image 1101b. More specifically, the orthodontic value acquisition unit 1105 may acquire orthodontic target direction information and orthodontic target distance information about each of the teeth by comparing vector information about each of the teeth included in the virtual orthodontic image 1103a with vector information about each of the teeth included in the malocclusion image 1101b. The orthodontic target direction information may be information about a direction in which the teeth in the malocclusion state are moved to be corrected into a corrected state. The orthodontic target distance information may be information about a direction in which the teeth in the malocclusion state are moved to be corrected into a corrected state.

According to one embodiment, the orthodontic value acquisition unit 1105 may acquire an orthodontic target value by calculating the orthodontic target direction information and the orthodontic target distance information based on a specified equation. The specified equation may vary depending on the company or institution operating the present invention. The orthodontic target value may be a value indicating a degree to which the teeth in the malocclusion state have to be moved as they are corrected in arrangement of the corrected state. That is, the orthodontic target value may be a target value by which each of the teeth based on the malocclusion image has to be moved to form each of the teeth in the corrected arrangement.

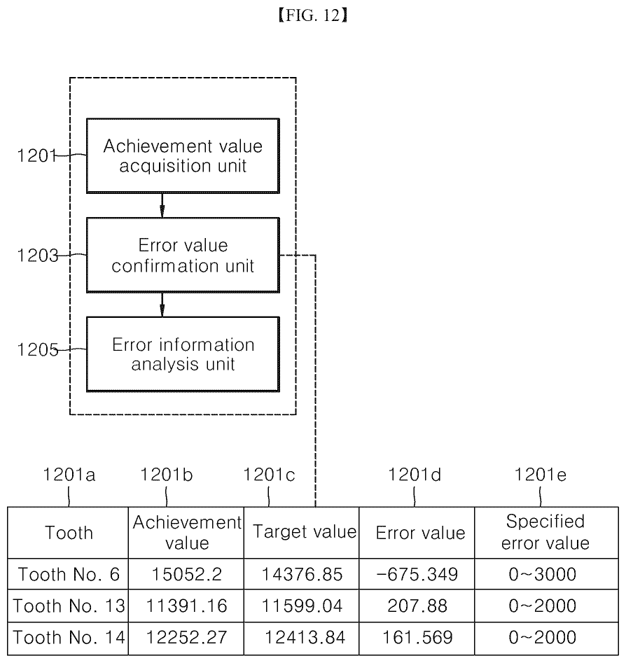

FIG. 12 is a block diagram for explaining an evaluation information provision unit of the apparatus for providing an orthodontic status and orthodontic treatment evaluation information based on tooth part scan data of a patient according to one embodiment of the present invention.

Referring to FIG. 12, the apparatus for providing an orthodontic status and orthodontic treatment evaluation information based on tooth part scan data of a patient (e.g., the apparatus 800 for providing an orthodontic status and treatment evaluation information based on tooth scan data of FIG. 8) (hereinafter, referred to as an apparatus for providing an orthodontic status and treatment evaluation information) may include an evaluation information provision unit 1200 (e.g., the evaluation information provision unit (805) of FIG. 8).

According to one embodiment, the evaluation information provision unit 1200 may include an achievement value acquisition unit 1201, an error value confirmation unit 1203, and an error information analysis unit 1205.

According to one embodiment, when the acquisition of the orthodontic completion image is completed through the orthodontic completion image acquisition unit (e.g., the orthodontic completion image acquisition unit 803 of FIG. 8), the achievement value acquisition unit 1201 may analyze the orthodontic completion image through a pre-stored malocclusion confirmation algorithm to acquire an orthodontic achievement value 1201b for each of the patient's corrected teeth. A detailed description related to the pre-stored malocclusion confirmation algorithm will be described with reference to FIG. 9.

According to one embodiment, when the acquisition of the orthodontic completion image for the patient's teeth arrangement in which the orthodontic treatment is completed by the transparent orthodontic appliance is completed, the achievement value acquisition unit 1201 may confirm a teeth arrangement state of the patient based on the orthodontic completion image through the pre-stored malocclusion confirmation algorithm. The achievement value acquisition unit 1201 may acquire the orthodontic achievement value 1201b for each of the teeth by confirming the teeth arrangement state of the patient based on the orthodontic completion image. The orthodontic achievement value 1201b is information including orthodontic achievement direction information and orthodontic achievement distance information, and may be vector information about each of the teeth. The orthodontic achievement value 1201b may be a value acquired by calculating the orthodontic achievement direction information and the orthodontic achievement distance information by a specified equation.

According to one embodiment, when the acquisition of the orthodontic achievement value 1201b is completed, the error value confirmation unit 1203 may compare an orthodontic target value 1201c (e.g., the orthodontic target value of FIG. 11) with the orthodontic achievement value 1201b. A detailed description related to the orthodontic target value 1201c will be described with reference to FIG. 11. The error value confirmation unit 1203 may compare the orthodontic target value 1201c with the orthodontic achievement value 1201b to acquire an error value 1201d of the orthodontic achievement value 1201b with respect to the orthodontic target value 1201c. The error value confirmation unit 1203 may determine whether the acquired error value 1201d is within a specified error value range 1201e.

A configuration of 1201 shown in FIG. 12 may be a record table stored by the error value confirmation unit 1203. For example, the error value confirmation unit 1203 may compare an orthodontic achievement value for tooth No. 13, which is acquired by the achievement value acquisition unit 201, with an orthodontic target value for tooth No. 13. The error value confirmation unit 1203 may acquire an error value of 207.88 by comparing the orthodontic achievement value of 11391.16 for tooth No. 13 with the orthodontic target value of 12252.27 for tooth No. 13. In this case, the error value confirmation unit 1203 may determine whether the acquired error value is within the specified error value. The specified error value may vary depending on each tooth and each type of malocclusion.

According to one embodiment, the error information analysis unit 1205 may generate error information based on a result determined according to performance of a function of the error value confirmation unit 1203. The error information may be information about whether an error value for each of the teeth is equal to or less than the specified error value. That is, the error information may include all information about the orthodontic achievement value, the orthodontic target value, the error value, and the specified error value for each of the teeth.

According to one embodiment, the error information analysis unit 1205 may generate orthodontic treatment evaluation information by analyzing the generated error information through the artificial intelligence solution generation algorithm. A detailed description related to the artificial intelligence solution generation algorithm will be given with reference to FIG. 9. The error information analysis unit 1206 may analyze the error information through the artificial intelligence solution generation algorithm. More specifically, the error information analysis unit 1205 may confirm that the orthodontic treatment for malocclusion has failed when it is confirmed that the error value is not within the specified error value based on the error information. In addition, the error information analysis unit 1205 may confirm that the orthodontic treatment for malocclusion succeeds when it is confirmed that the error value is within the specified error value based on the error information.

According to one embodiment, the error information analysis unit 1205 may confirm whether the orthodontic treatment for each of the patient's teeth succeeds based on the error information, and may generate orthodontic treatment evaluation information based on the confirmation result.

According to one embodiment, the error information analysis unit 1205 may generate orthodontic treatment evaluation information notifying that the orthodontic treatment for malocclusion is not properly performed, when it is confirmed that the error value is not within the specified error value based on the error information. In this case, the error information analysis unit 1205 may derive cause information in which the error value is not within the specified error value range through the artificial intelligence solution generation algorithm, and may generate orthodontic improvement information for solving a cause based on the cause information. The orthodontic improvement information is information generated through the artificial intelligence solution generation algorithm, and may be exemplary treatment information derived based on orthodontic treatment history information about another patient learned through the artificial intelligence solution generation algorithm. That is, the error information analysis unit 1205 may cause the exemplary treatment information to present an orthodontic guide for the teeth whose orthodontic treatment has failed.

According to one embodiment, the error information analysis unit 1205 may generate orthodontic treatment evaluation information notifying that the orthodontic treatment for malocclusion is properly performed, when it is confirmed that the error value is within the specified error value based on the error information. In this case, the error information analysis unit 1205 may derive supplementary point information about each of the corrected teeth through the artificial intelligence solution generation algorithm. For example, the supplementary point information may include information about a recommended wearing period of the transparent orthodontic appliance or attention point information in order to completely wear each of the teeth on the orthodontic position.

According to one embodiment, the error information analysis unit 1205 may confirm an error value based on the error information, and may confirm which of the specified error value ranges the confirmed error value is within. The specified error value range may include at least two or more state ranges. For example, the specified error value range may include an even number range, a good range, and an additional treatment recommendation range. According to one embodiment, the error information analysis unit 1205 may generate orthodontic treatment evaluation information notifying that the orthodontic treatment for malocclusion is effectively performed, when it is confirmed that the error value is within the good range. As another example, the error information analysis unit 1205 may determine that the orthodontic treatment for malocclusion is performed based on the treatment solution information, but insufficient parts for the orthodontic treatment occur, when it is confirmed that the error value is within the treatment recommendation range. Accordingly, when the orthodontic treatment evaluation information is generated, the error information analysis unit 1205 may analyze the insufficient parts through the artificial intelligence solution generation algorithm, may acquire treatment supplementation information for supplementing the insufficient parts, and may include the treatment supplementation information in the orthodontic treatment evaluation information.

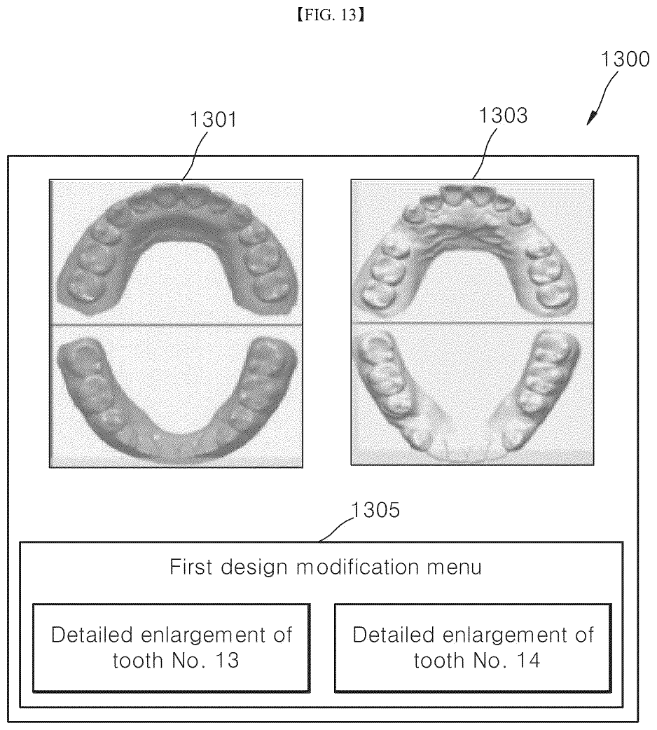

FIG. 13 is a view for explaining an interface of the apparatus for providing an orthodontic status and orthodontic treatment evaluation information based on tooth part scan data of a patient according to one embodiment of the present invention.

Referring to FIG. 13, the apparatus for providing an orthodontic status and orthodontic treatment evaluation information based on tooth part scan data (e.g., the apparatus 800 for providing an orthodontic status and treatment evaluation information based on tooth scan data of FIG. 8) (hereinafter, referred to as an apparatus for providing an orthodontic status and treatment evaluation information), which is implemented by a computing device including one or more processors and one or more memories storing instructions executable by the processors, may include an evaluation information provision unit (e.g., the evaluation information provision unit (805) of FIG. 8).

According to one embodiment, the evaluation information provision unit may include an orthodontic appliance design generation unit (not shown) and an orthodontic appliance design provision unit.

According to one embodiment, the evaluation information provision unit may allow the orthodontic appliance design generation unit to generate a first design, which is design information about the transparent orthodontic appliance based on the exemplary treatment information, when the orthodontic treatment evaluation information including the exemplary treatment information is provided to the medical personnel account.

According to one embodiment, the orthodontic appliance design generation unit may generate the first design, which is design information about teeth arrangement of a patient whose orthodontic treatment is completed, based on the exemplary treatment information. In this case, the generated first design may be design information for manufacturing a new transparent orthodontic appliance (e.g., a second transparent orthodontic appliance) in order to supplement the insufficient parts for the orthodontic treatment after the patient's initial teeth arrangement is corrected by a transparent orthodontic appliance (e.g., a first transparent orthodontic appliance).

According to one embodiment, when the generation of the first design is completed, the orthodontic appliance design provision unit may acquire second design information, which is a design of the transparent orthodontic appliance based on the orthodontic target value, and may provide the medical person account with an interface 1300 that may compare the first design with the second design. That is, the orthodontic appliance design provision unit may provide the medical personnel account with the interface 1300 that may visually confirm a first transparent orthodontic appliance 1301 based on the second design and a second transparent orthodontic appliance 1303 based on the first design.

According to one embodiment, while images for the first transparent orthodontic appliance and the second transparent orthodontic appliance are provided to the medical personnel account through the interface 1300, when doctor opinion information for changing the design of the second transparent orthodontic appliance is received from the medical personnel account, the evaluation information provision unit may modify the first design based on the received doctor opinion information. The doctor opinion information may be design modification information for modifying the first design. That is, a user of the medical personnel account may confirm the patient's teeth arrangement, and may change the design based on the first design by inputting the doctor opinion information to the interface when a change for the design is additionally required.

A configuration of 1305 in FIG. 13 is a menu for modifying the first design, and may be one of functions included in the interface 1300. The user of the medical personnel account may enlarge an image for each tooth in detail through the configuration of 1305. The evaluation information provision unit may modify the position and shape of the enlarged tooth based on the received doctor opinion information when receiving the doctor opinion information, which is input information for modifying the enlarged tooth, from the medical personnel account in a state in which the image for the tooth is enlarged in detail. In this case, the tooth included in the image is a 3D modeling image, in which the 3D modeling image may be an image that may be acquired when the tooth image is analyzed through the pre-stored malocclusion confirmation algorithm.

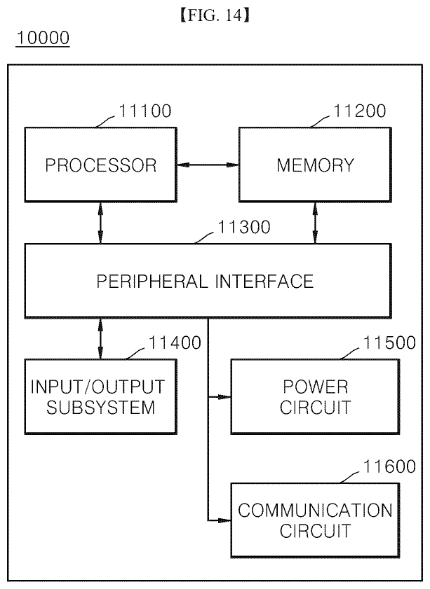

FIG. 14 is a view showing one example of an internal configuration of a computing device according to one embodiment of the present invention.

FIG. 14 is a view showing an example of an internal configuration of the computing device according to one embodiment of the present invention. In the following description, redundant descriptions of the embodiment corresponding to the above descriptions for FIGS. 1 to 13 will be omitted.

As shown in FIG. 14, a computing device 10000 may at least include at least one processor 11100, a memory 11200, a peripheral interface 11300, an input/output (I/O) subsystem 11400, a power circuit 11500, and a communication circuit 11600. In this case, the computing device 10000 may correspond to a user terminal A connected to a tactile interface device or correspond to the above-described computing device B.

The memory 11200 may include, for example, a high-speed random access memory, a magnetic disk, an SRAM, a DRAM, a ROM, a flash memory, or a non-volatile memory. The memory 11200 may include a software module, an instruction set, or other various data necessary for the operation of the computing device 10000.

In this case, access to the memory 11200 from other components of the processor 11100 or the peripheral interface 11300, may be controlled by the processor 11100.

The peripheral interface 11300 may combine an input and/or output peripheral device of the computing device 10000 to the processor 11100 and the memory 11200. The processor 11100 may execute the software module or the instruction set stored in the memory 11200, thereby performing various functions for the computing device 10000 and processing data.

The input/output subsystem 11400 may combine various input/output peripheral devices to the peripheral interface 11300. For example, the input/output subsystem 11400 may include a controller for combining the peripheral device such as monitor, keyboard, mouse, printer, or a touch screen or sensor, if needed, to the peripheral interface 11300. According to another aspect, the input/output peripheral devices may be combined to the peripheral interface 11300 without passing through the input/output subsystem 11400.

The power circuit 11500 may provide power to all or a portion of the components of the terminal. For example, the power circuit 11500 may include a power failure detection circuit, a power converter or inverter, a power status indicator, a power failure detection circuit, a power converter or inverter, a power status indicator, or arbitrary other components for generating, managing, or distributing power.

The communication circuit 11600 may use at least one external port to enable communication with other computing devices.

Alternatively, as described above, the communication circuit 11600 may include an RF circuit, if needed, to transmit and receive an RF signal, also known as an electromagnetic signal, thereby enabling communication with other computing devices.

The above embodiment of FIG. 14 is merely an example of the computing device 10000, and the computing device 11000 may have a configuration or arrangement in which some components shown in FIG. 14 are omitted, additional components not shown in FIG. 14 are further provided, or at least two components are combined. For example, a computing device for a communication terminal in a mobile environment may further include a touch screen, a sensor, or the like, in addition to the components shown in FIG. 14. The communication circuit 11600 may include a circuit for RF communication of various communication schemes (such as WiFi, 3G, LTE, Bluetooth, NFC, and Zigbee). The components that may be included in the computing device 10000 may be implemented by hardware, software, or a combination of both hardware and software which include at least one integrated circuit specialized in a signal processing or an application.

The methods according to the embodiments of the present invention may be implemented in the form of program instructions to be executed through various computing devices so as to be recorded in a computer-readable medium. In particular, a program according to the embodiment of the present invention may be configured as a PC-based program or an application dedicated to a mobile terminal. The application to which the present invention is applied may be installed in a user terminal through a file provided by a file distribution system. For example, a file distribution system may include a file transmission unit (not shown) that transmits the file according to the request of the user terminal.

The above-described device may be implemented by hardware components, software components, and/or a combination of hardware components and software components. For example, the devices and components described in the embodiments may be implemented by using at least one general purpose computer or special purpose computer such as a processor, a controller, an arithmetic logic unit (ALU), a digital signal processor, a microcomputer, a field programmable gate array (FPGA), a programmable logic unit (PLU), a microprocessor, or any other device capable of executing and responding to instructions. The processing device may execute an operating system (OS) and at least one software application executed on the operating system. In addition, the processing device may access, store, manipulate, process, and generate data in response to the execution of the software. For the further understanding, in some cases, one processing device may be used, however, those skilled in the art will be appreciated that the processing device may include a plurality of processing elements and/or a plurality of types of processing elements. For example, the processing device may include a plurality of processors or one processor and one controller. In addition, other processing configurations, such as a parallel processor, are also possible.

The software may include a computer program, a code, an instruction, or a combination of at least one thereof, may configure the processing device to operate as desired, or may instruct the processing device independently or collectively. In order to be interpreted by the processor or to provide instructions or data to the processor, the software and/or data may be permanently or temporarily embodied in any type of machine, component, physical device, virtual equipment, and computer storage medium or device. The software may be distributed over computing devices connected to networks, so as to be stored or executed in a distributed manner. The software and data may be stored in at least one computer-readable recording medium.

The above-described embodiments of the present invention may be recorded in computer-readable media including program instructions to implement various operations embodied by a computer. The computer-readable medium may also include, alone or in combination with the program instructions, data files, data structures, and the like. The program instructions recorded on the media may be those specially designed and constructed for the purposes of the embodiments, or they may be of the kind well-known and available to those having skill in the computer software arts. Examples of computer-readable recording media include magnetic media such as hard disks, floppy disks, and magnetic tape; optical media such as CD ROM disks and DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules in order to perform the operations of the above-described embodiments of the present invention, or vice versa.

While the embodiments have been described with reference to limited examples and drawings as described above, it will be apparent to one of ordinary skill in the art that various changes and modifications may be made from the above description. Suitable results may be achieved if the described techniques are performed in a different order, and/or if components in a described system, architecture, device, or circuit are combined in a different manner, and/or replaced or supplemented by other components or their equivalents. Therefore, other implementations, other embodiments, and equivalents of the claims are within the scope of the following claims.

1. A method for providing an orthodontic status and orthodontic treatment evaluation information based on tooth part scan data of a patient, which is implemented by a computing device including one or more processors and one or more memories storing instructions executable by the processors, the method comprising:
an initial image acquisition step of acquiring a first tooth image, which is an image for patient's teeth arrangement, based on received first tooth part scan data when first tooth part scan data, which is three-dimensional scan data acquired by capturing a patient's head, is received;
an orthodontic image acquisition step of, when the acquisition of the first tooth part image is completed, confirming a teeth arrangement state based on the first tooth image through a pre-stored algorithm, acquiring treatment solution information for correcting the teeth arrangement based on the confirmed teeth arrangement state, and acquiring a second tooth image, which is an image for predicted teeth arrangement upon orthodontic completion based on the acquired treatment solution information;
an orthodontic appliance design generation step of, when the acquisition of the second tooth image is completed, generating a design of a transparent orthodontic appliance for correcting the patient's teeth arrangement into teeth arrangement corresponding to the second tooth image;
an intermediate image acquisition step of acquiring a third tooth image, which is an image for arrangement of patient's teeth being corrected, based on received second tooth part scan data when second tooth part scan data, which is new three-dimensional scan data, is received in a process of correcting the patient's teeth arrangement as the patient wears the transparent orthodontic appliance based on the generated design; and
an orthodontic status information provision step of, when the acquisition of the third tooth image is completed, acquiring tooth movement vector information about the patient through the first tooth image, the second tooth image, and the third tooth image to generate orthodontic status information for orthodontic treatment of the patient based on the acquired tooth movement vector information to provide the orthodontic status information to a medical personnel account.

2. The method of claim 1, wherein the orthodontic image acquisition step includes:
a process start step of starting a malocclusion confirmation process when the acquisition of the first tooth image is completed;
a malocclusion classification step of, when the malocclusion confirming process starts, acquiring teeth arrangement state information about the patient by analyzing the first tooth image through the pre-stored algorithm to classify the acquired teeth arrangement state information as one of a plurality of malocclusion type information; and
a solution information acquisition step of, when the teeth arrangement state information is classified as one of the plurality of malocclusion type information, acquiring treatment solution information about the classified malocclusion type information through a machine learning-based artificial intelligence solution generation algorithm that derives a solution for orthodontic treatment.

3. The method of claim 1, wherein in the orthodontic status information provision step, tooth movement vector information about each of the patient's teeth is acquired based on a common point included in a first cephalometric image corresponding to the first tooth part scan image and a second cephalometric image corresponding to the second tooth part scan data.

4. The method of claim 3, wherein the common point is a common location located on a patient's cephalic part included in the first cephalometric image and the second cephalometric image, in which the common point includes at least three locations that are not changed even when the patient's teeth arrangement is corrected, and serves as a reference point for overlapping the first tooth image, the second tooth image, and the third tooth image.

5. The method of claim 3, wherein the orthodontic status information provision step includes:
an image overlapping step of generating a prognostic image by overlapping the first tooth image, the second tooth image, and the third tooth image based on the common point included in the first cephalometric image and the second cephalometric image;
an orthodontic progress confirmation step of, when the generation of the prognostic image is completed, generating first tooth movement vector information including first tooth movement direction information and first tooth movement distance information based on the prognostic image by confirming a direction and distance in which each of the teeth is moved by comparing teeth arrangement corresponding to the first tooth image with teeth arrangement corresponding to the third tooth image;
an orthodontic progress prediction step of, when a function of the orthodontic progress confirmation step is performed, generating second tooth movement vector information including second tooth movement direction information and second tooth movement distance information based on the prognostic image by confirming a direction and distance in which each of the teeth is expected to be moved by comparing the teeth arrangement corresponding to the first tooth image with teeth arrangement corresponding to the second tooth image; and
an information generation step of, when the acquisition of the first tooth movement vector information and the second tooth movement vector information is completed, starting an information generation process by generating orthodontic status information based on the first tooth movement vector information and the second tooth movement vector information.

6. The method of claim 5, wherein in the image overlapping step, the prognostic image is generated by applying a graphic effect such that the first tooth image, the second tooth image, and the third tooth image, which overlap each other based on the common point, are visually distinguished.

7. The method of claim 5, wherein the orthodontic progress prediction step includes:
a fourth tooth image acquisition step of, when the direction and distance in which each of the teeth is expected to be moved, generating a plurality of fourth tooth images corresponding to each of a plurality of time points based on the treatment solution information from the first tooth image and the second tooth image; and
a second tooth movement vector information generation step of, when the generation of the plurality of fourth tooth images is completed, generating the second tooth movement vector information about teeth arrangement included in each of the plurality of fourth tooth images by comparing each of the plurality of fourth tooth images in order of progress, and
each of the plurality of time points is at least two time points input by the medical personnel account.

8. The method of claim 7, wherein the information generation step includes:
a direction confirmation step of, when the generation of the first tooth movement vector information and the second tooth movement vector information is completed, confirming that an error rate of a movement axis direction based on the first tooth movement direction information with respect to a movement axis direction based on the second tooth movement direction information is equal to or less than a specified error rate by comparing the second tooth movement direction information with the first tooth movement direction information;
a distance confirmation step of confirming that an error rate of a movement distance based on the first tooth movement distance information with respect to a movement distance based on the second tooth movement distance information is equal to or less than the specified error rate by comparing the second tooth movement distance information with the first tooth movement distance information during the direction confirmation step; and
an orthodontic status information generation step of, when result information based on the direction confirmation step and the distance confirmation step is acquired, generating the orthodontic status information indicating a status of orthodontic treatment for the patient's teeth arrangement based on the result information.

9. The method of claim 1, wherein the method for providing an orthodontic status and orthodontic treatment evaluation information based on tooth part scan data of a patient further comprises: an orthodontic treatment evaluation information provision step, and
the orthodontic treatment evaluation information provision step includes:
a target value acquisition step of, when a function of the initial image acquisition step is completed, acquiring a malocclusion image for patient's teeth based on the first tooth part scan data, acquiring treatment solution information based on the acquired malocclusion image, and acquiring an orthodontic target value for correcting the patient's malocclusion based on the acquired treatment solution information;
an orthodontic completion image acquisition step of, in a state in which the acquisition of the orthodontic target value is completed, acquiring an orthodontic completion image that is an image for corrected teeth arrangement based on the third tooth part scan data when third tooth part scan data, which is new three-dimensional scan data acquired by capturing a patient who has completed the orthodontic treatment by the transparent orthodontic appliance; and
an evaluation information provision step of acquiring an orthodontic achievement value for each of the corrected teeth based on the acquired orthodontic completion image, and when error information is acquired by comparing the orthodontic achievement value with the orthodontic target value, generating orthodontic treatment evaluation information, which is evaluation information about the orthodontic treatment, based on the acquired error rate to provide the orthodontic treatment evaluation information to the medical personnel account.

10. The method of claim 9, wherein the target value acquisition step includes:
a malocclusion confirmation start step of starting a malocclusion confirmation process when the first tooth part scan data is received from the medical personnel account;
a malocclusion determination step of, when the malocclusion confirmation process is started, determining patient's malocclusion by analyzing the malocclusion image through a pre-stored malocclusion confirmation algorithm to confirm the patient's teeth arrangement through the analyzed malocclusion image, and classifying the confirmed teeth arrangement as any one of a plurality of malocclusion information; and
a solution acquisition step of, when the determination on the patient's malocclusion is completed, acquiring treatment solution information about the patient's malocclusion through a machine learning-based artificial intelligence solution generation algorithm that derives a solution for orthodontic treatment.

11. The method of claim 10, wherein in the malocclusion determination step, when the patient's teeth arrangement is confirmed, at least one of a position, a contact relationship between adjacent teeth, a vertical relationship, rotation, and inclination for each of the patient's teeth included in the malocclusion image is confirmed through the pre-stored malocclusion confirmation algorithm.

12. The method of claim 11, wherein the target value acquisition step includes:
a guide application step of, when the acquisition of the treatment solution information is completed by performing a function of the solution acquisition step, applying an orthodontic guide based on the treatment solution information to the malocclusion image through the solution generation algorithm;
a virtual orthodontic image acquisition step of, as the orthodontic guide based on the treatment solution information is applied to the malocclusion image, acquiring a virtual orthodontic image, which is a virtual image corresponding to teeth arrangement in which the patient's orthodontic treatment is completed, by arranging each of the patient's teeth so as to be in a state in which the patient's orthodontic treatment is completed; and
an orthodontic value acquisition step of, when the acquisition of the virtual orthodontic image is completed, acquiring orthodontic target direction information and orthodontic target distance information about each of the teeth by comparing the virtual orthodontic image with the malocclusion image to acquire the orthodontic target value, which is a reference value for correcting the patient's malocclusion.

13. The method of claim 12, wherein the evaluation information provision step includes:
an achievement value acquisition step of, when the orthodontic completion image is acquired by performing a function of the orthodontic completion image acquisition step, acquiring the orthodontic achievement value for each of the corrected patient's teeth by analyzing the orthodontic completion image through the pre-stored malocclusion confirmation algorithm;
an error value confirmation step of, when the acquisition of the orthodontic achievement value is completed, determining whether the acquired error value is within a range of a specified error value by comparing the orthodontic target value with the orthodontic achievement value to acquire an error value of the orthodontic achievement value for the orthodontic target value; and
an error information analysis step of, based on a result determined according to a function of performing the error value confirmation step, generating the orthodontic treatment evaluation information by generating error information and analyzing the generated error information through the artificial intelligence solution generation algorithm.

14. The method of claim 13, wherein the orthodontic treatment evaluation information is information obtained by determining whether or not orthodontic treatment for each of the patient's teeth is successful based on the error information, and includes orthodontic improvement information for correcting each of the teeth that has failed the orthodontic treatment when it is determined that the orthodontic treatment for each of the patient's teeth based on the error information has failed, and
the orthodontic improvement information is information generated through the artificial intelligence solution generation algorithm, and is exemplary treatment information derived based on orthodontic treatment history information about another patient learned through the artificial intelligence solution generation algorithm.

15. The method of claim 14, wherein the evaluation information provision step includes:
an orthodontic appliance design generation step of, when the orthodontic treatment evaluation information including the exemplary treatment information is provided to the medical personnel account, generating a first design, which is design information about the transparent orthodontic appliance based on the exemplary treatment information; and
an orthodontic appliance design provision step of, when the generation of the first design is completed, providing an interface capable of comparing the first design with the second design to the medical personnel account by acquiring second design information, which is design information about the transparent orthodontic appliance based on the orthodontic target value.

16. The method of claim 15, wherein when doctor opinion information for changing the design of the transparent orthodontic appliance is received from the medical personnel account, the interface modifies the first design based on the received doctor opinion information.

17. An apparatus for providing an orthodontic status and orthodontic treatment evaluation information based on tooth part scan data of a patient, which includes a computing device including one or more processors and one or more memories storing instructions executable by the processors, the apparatus comprising:
an initial image acquisition unit which acquires a first tooth image, which is an image for patient's teeth arrangement, based on received first tooth part scan data when first tooth part scan data, which is three-dimensional scan data acquired by capturing a patient's head, is received;
an orthodontic image acquisition unit which, when the acquisition of the first tooth part image is completed, confirms a teeth arrangement state based on the first tooth image through a pre-stored algorithm, acquires treatment solution information for correcting the teeth arrangement based on the confirmed teeth arrangement state, and acquires a second tooth image, which is an image for predicted teeth arrangement upon orthodontic completion based on the acquired treatment solution information;
an orthodontic appliance design generation unit which, when the acquisition of the second tooth image is completed, generates a design of a transparent orthodontic appliance for correcting the patient's teeth arrangement into teeth arrangement corresponding to the second tooth image;
an intermediate image acquisition unit which acquires a third tooth image, which is an image for arrangement of patient's teeth being corrected, based on received second tooth part scan data when second tooth part scan data, which is new three-dimensional scan data, is received in a process of correcting the patient's teeth arrangement as the patient wears the transparent orthodontic appliance based on the generated design; and
an orthodontic status information provision unit which, when the acquisition of the third tooth image is completed, acquires tooth movement vector information about the patient through the first tooth image, the second tooth image, and the third tooth image to generate orthodontic status information for orthodontic treatment of the patient based on the acquired tooth movement vector information to provide the orthodontic status information to a medical personnel account.

18. A computer-readable recording medium that stores instructions for allowing a computing device to perform the following steps, wherein the steps comprise:
an initial image acquisition step of acquiring a first tooth image, which is an image for patient's teeth arrangement, based on received first tooth part scan data when first tooth part scan data, which is three-dimensional scan data acquired by capturing a patient's head, is received;
an orthodontic image acquisition step of, when the acquisition of the first tooth part image is completed, confirming a teeth arrangement state based on the first tooth image through a pre-stored algorithm, acquiring treatment solution information for correcting the teeth arrangement based on the confirmed teeth arrangement state, and acquiring a second tooth image, which is an image for predicted teeth arrangement upon orthodontic completion based on the acquired treatment solution information;
an orthodontic appliance design generation step of, when the acquisition of the second tooth image is completed, generating a design of a transparent orthodontic appliance for correcting the patient's teeth arrangement into teeth arrangement corresponding to the second tooth image;
an intermediate image acquisition step of acquiring a third tooth image, which is an image for arrangement of patient's teeth being corrected, based on received second tooth part scan data when second tooth part scan data, which is new three-dimensional scan data, is received in a process of correcting the patient's teeth arrangement as the patient wears the transparent orthodontic appliance based on the generated design; and
an orthodontic status information provision step of, when the acquisition of the third tooth image is completed, acquiring tooth movement vector information about the patient through the first tooth image, the second tooth image, and the third tooth image, and generating orthodontic status information for orthodontic treatment of the patient based on the acquired tooth movement vector information to provide the orthodontic status information to a medical personnel account.
